(19) United States
(12) Patent Application Publication
(10) Pub. No.: US 2007/0259345 A1
(43) Pub. Date: Nov. 8, 2007

(54) Target determination using compound probes

(75) Inventor: Nicholas M. Sampas, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Loveland, CO (US)

(21) Appl. No.: 11/417,324

(22) Filed: May 3, 2006

(51) Int. Cl. C12Q 1/68; C12M 3/00

(52) U.S. Cl. 435/6; 435/287.2

(57) ABSTRACT

The present invention generally relates to systems and methods for identifying and/or quantifying targets such as nucleic acid targets using compound probes (e.g., oligonucleotide probes), which can comprise first and second sequences able to hybridize to one or more nucleic acids or portions thereof. In one aspect, a nucleic acid (e.g., one or more genomes or chromosomes) may be fragmented, and the fragments exposed to a compound probe. By suitably labeling of the fragments, the amount of binding of each of the fragments to the compound probe may be determined. In some cases, the fragments may be distinguishably labeled, and in certain embodiments, the fragments may be sorted (e.g., via size or charge) prior to exposure to the compound probe.

### BACKGROUND

Arrays of nucleic acids have become an increasingly important tool in the biotechnology industry and related fields. These nucleic acid arrays, in which a plurality of distinct or different nucleic acids are positioned on a solid support surface in the form of an array or pattern, find use in a variety of applications, including gene expression analysis, nucleic acid synthesis, drug screening, nucleic acid sequencing, mutation analysis, array CGH, location analysis (also known as ChIP-Chip), and the like.

Arrays having a large number of spots are advantageous in that large genomes or transcriptomes can be assayed at higher resolutions and/or with fewer number of slides per experiment. Current methods of increasing the density of spots per array include forming spots with smaller surface areas and/or positioning spots closer together on the array. Although these methods may be useful, other methods of increasing the effective probe density of arrays would be beneficial.

### SUMMARY OF THE INVENTION

The present invention generally relates to systems and methods for identifying and/or quantifying targets such as nucleic acid targets using compound probes (e.g., oligonucleotide probes) and the like. The subject matter of the present invention involves, in some cases, interrelated products, alternative solutions to a particular problem, and/or a plurality of different uses of one or more systems and/or articles.

One aspect of the invention is directed to a method. In one set of embodiments, the method includes acts of providing an oligonucleotide probe, exposing the oligonucleotide probe to a first nucleic acid labeled with a first detection entity such that at least a portion of the first nucleic acid hybridizes to a first sequence of the oligonucleotide probe, and exposing the oligonucleotide probe to a second nucleic acid, different from the first nucleic acid and labeled with a second detection entity, such that least a portion of the second nucleic acid hybridizes to a second sequence of the oligonucleotide probe. In some cases, the act of exposing the exposing the oligonucleotide probe to the first nucleic acid and the act of exposing the oligonucleotide probe to the second nucleic acid occurs simultaneously. The precursor nucleic acid may be a genome or a chromosome in certain instances.

In one embodiment, the method also includes an act of cleaving a precursor nucleic acid to produce the first nucleic acid and the second nucleic acid. The act of cleaving the precursor nucleic acid includes, in some cases, exposing the precursor nucleic acid to a restriction endonuclease. In some embodiments, the method also includes an act of labeling the precursor nucleic acid with a first detection entity and a second detection entity prior to cleaving the precursor nucleic acid.

In another embodiment, the method also includes acts of separating the first nucleic acid and the second nucleic acid, and thereafter, labeling the first nucleic acid with the first detection entity and the second nucleic acid with the second detection entity. The act of separating may comprise, in some instances, separating the first nucleic acid and the second nucleic acid using gel electrophoresis, capillary electrophoresis, chromatography, HPLC, mass separation, or flow cytometry.

The oligonucleotide probe is immobilized relative to a surface in some embodiments. In certain cases, the oligonucleotide probe can have a length of at least 60 nucleotides, at least 80 nucleotides, or at least 100 nucleotides. In one embodiment, the first sequence and the second sequence of the oligonucleotide probe are separated by a linker segment.

In some embodiments, the first detection entity is fluorescent, and/or the first detection entity may comprise a dye, a nucleic acid analog, or an antibody. The method may also thus include an act of determining association of the first and second detection entities with the oligonucleotide probe. For instance, the association of the first and second detection entities may be determined using fluorescence.

In various embodiments, the first sequence of the oligonucleotide probe can have a length of at least 50, 75, or 100 nucleotides. The method, in certain instances, may include an act of providing a plurality of oligonucleotide probes, at least some of which are non-identical, and exposing the plurality of oligonucleotide probes to the first and second nucleic acids, and in some cases, providing providing at least 100, 1,000, 10,000, or 100,000 non-identical oligonucleotide probes. In some embodiments, the plurality of oligonucleotide probes are each immobilized relative to a surface at a density of at least about 0.01 pmol/mm², at least about 0.03 pmol/mm², at least about 0.1 pmol/mm², at least about 0.3 pmol/mm², or at least about 1 pmol/mm².

In one embodiment, the method also includes an act of identifying at least one of the first or second nucleic acids. The method, in some cases, may also include an act of comparing hybridization of the first nucleic acid with the oligonucleotide probe, and hybridization of the second nucleic acid with the oligonucleotide probe, for instance, to determine a ratio of concentration of the first nucleic acid to the second nucleic acid, and/or to determine a concentration of at least one of the first or second nucleic acids. The method can also include, in one embodiment, an act of identifying one or more restriction sites within the precursor nucleic acid by determining hybridization of the first nucleic acid with the oligonucleotide probe, and hybridization of the second nucleic acid with the oligonucleotide probe.

In another set of embodiments, the method includes the acts of determining binding of a first chromosome to each of first and second oligonucleotide probes, determining binding of a second chromosome to each of the first and second oligonucleotide probes, and determining translocation between the first and second chromosomes based on hybridization of each of the first and second chromosomes to each of the first and second oligonucleotide probes.

In yet another set of embodiments, the method includes acts of cleaving a nucleic acid into a plurality of nucleic acid fragments, separating the nucleic acid fragments into at least a first sample and a second sample, labeling at least some of the fragments of the first sample with a first detection entity and at least some of the fragments of the second sample with a second detection entity, and exposing at least some of the nucleic acid fragments of the first and second samples to at least one compound probe. In still another set of embodiments, the method includes acts of cleaving a nucleic acid into a plurality of nucleic acid fragments, each having a first end and a second end, labeling the first end of at least some of the nucleic acid fragments with a first detection label and the second end of at least some of the nucleic acid fragments with a second detection label, and exposing at least some of the nucleic acid fragments to at least one compound probe.

The method, according to yet another set of embodiments, includes acts of labeling one or more portions of a first chromosome with a first detection entity and one or more portions of a second chromosome with a second detection entity, fragmenting each of the first chromosome and the second chromosome to produce a plurality of chromosome fragments, and exposing the chromosome fragments to one or more compound probes. The acts of the method may be performed in the order recited. In some embodiments, the act of fragmenting each of the first and second chromosomes can occur before the act of labeling each of the first and second chromosomes.

In some cases, at least some of the one or more compound probes comprises a first sequence able to hybridize the first chromosome but not the second chromosome, and a second sequence able to hybridize the second chromosome but not the first chromosome. For instance, the first sequence may be able to hybridize the first chromosome but not other chromosomes, and the second sequence may be able to hybridize the second chromosome but not other chromosomes.

In another embodiment, the method may further include an act of determining whether a compound probe of the one or more compound probes has hybridized to both a portion of a first chromosome and a portion of a second chromosome, for instance, by identifying a translocation between the first and second chromosomes based on hybridization of the compound probe.

The invention is directed to an article, in another aspect. The article includes a composition in one set of embodiments, which is constructed and arranged to be used in an assay of a sample comprising a first nucleic acid labeled with a first detection entity and a second nucleic acid, different from the first nucleic acid and labeled with a second detection entity. In some cases, the composition includes an oligonucleotide probe able to hybridize to at least portions of each of the first nucleic acid and the second acid. In certain instances, each of the first detection entity and the second detection entity can be determined.

In another set of embodiments, the article may include a composition comprising a first oligonucleotide probe comprising a first nucleotide sequence able to hybridize to a first portion of a first chromosome and a second nucleotide sequence able to hybridize to a second portion of the first chromosome, and a second oligonucleotide probe comprising a first nucleotide sequence able to hybridize to a first portion of a second chromosome and a second nucleotide sequence able to hybridize to a second portion of the second chromosome.

In still another set of embodiments, the article includes an array comprising an oligonucleotide probe able to hybridize to at least portions of each of a first nucleic acid labeled with a first detection entity and a second nucleic acid, different from the first nucleic acid, labeled with a second detection entity. In some cases, each of the first detection entity and the second detection entity can be determined using the array.

The article, in yet another set of embodiments, includes an array comprising a plurality of oligonucleotide probes, at least some of which each comprise a first nucleotide sequence able to hybridize to a first portion of a chromosome and a second nucleotide sequence able to hybridize to a second portion of the chromosome. In some cases, the array may comprise a first oligonucleotide probe comprising a first nucleotide sequence able to hybridize to a first portion of a first chromosome and a second nucleotide sequence able to hybridize to a second portion of the first chromosome, and a second oligonucleotide probe comprising a first nucleotide sequence able to hybridize to a first portion of a second chromosome and a second nucleotide sequence able to hybridize to a second portion of the second chromosome.

Kits are provided according to another aspect of the invention. In one set of embodiments, the kit includes a first oligonucleotide probe comprising a first nucleotide sequence able to hybridize to a first portion of a first chromosome and a second nucleotide sequence able to hybridize to a second portion of the first chromosome, and a second oligonucleotide probe comprising a first nucleotide sequence able to hybridize to a first portion of a second chromosome and a second nucleotide sequence able to hybridize to a second portion of the second chromosome. In another set of embodiments, the kit includes an oligonucleotide probe able to hybridize to at least portions of each of a first nucleic acid labeled with a first detection entity and a second nucleic acid, different from the first nucleic acid, labeled with a second detection entity, such that each of the first detection entity and the second detection entity can be determined

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A and 1B are schematic diagrams of first and second oligonucleotide probes including first and second nucleotide sequences, respectively, attached to an array surface (prior art);

FIG. 1C is a schematic diagram of a microarray including a plurality of spots comprising the oligonucleotide probes of FIGS. 1A and 1B (prior art);

FIGS. 2A-2F are schematic diagrams of different compound probes according to one embodiment of the invention;

FIGS. 3A-3F are schematic diagrams of oligonucleotide probes hybridized to target nucleotides sequences in a nucleic acid molecule of interest according to another embodiment of the invention;

FIG. 4 is a schematic diagram of a microarray including a plurality of spots comprising compound probes according to another embodiment of the invention;

FIGS. 5A-5C illustrate first and second oligonucleotide probes (FIGS. 5A-5B) that may be used in the microarray of FIG. 5C according to another embodiment of the invention;

FIG. 6 shows an example of a substrate carrying an array, in accordance with one embodiment of the invention;

FIG. 7 shows an enlarged view of a portion of FIG. 6;

FIG. 8 shows an enlarged view of another portion of the substrate of FIG. 6;

FIG. 9 shows an example of the use of a compound probe according to one embodiment of the invention;

FIG. 10 shows another example of the use of a compound probe according to another embodiment of the invention; and

FIGS. 11A-11B are schematic diagrams illustrating the use of a compound probe to determine translocation, according to one embodiment of the invention.

### DETAILED DESCRIPTION

The present invention generally relates to systems and methods for identifying and/or quantifying targets such as nucleic acid targets using various probes such as compound probes, which can comprise first and second sequences able to hybridize to one or more nucleic acids or portions thereof. In one aspect, a nucleic acid (e.g., one or more genomes or chromosomes) may be fragmented, and the fragments exposed to a compound probe. By suitably labeling of the fragments, the amount of binding of each of the fragments to the compound probe may be determined. In some cases, the fragments may be distinguishably labeled, and in certain embodiments, the fragments may be sorted (e.g., via size or charge) prior to exposure to the compound probe.

Certain aspects of the invention involve the analysis of nucleotide sequences of nucleic acid molecules using multiple probes per spot of an array or other substrate. In one embodiment, spots on an array may include long probes (e.g., probes comprising greater than about 60 base pairs). These probes may be in the form of compound probes (discussed below), which comprise at least first and second probes, including first and second nucleotide sequences capable of hybridizing to first and second target nucleotide sequences, respectively, in a nucleic acid molecule of interest. As such, a single spot of an array may include one or several different probes, which can increase the effective probe density of an array. The use of such multiple probes (i.e., probes having different nucleic acid sequences), which may include compound probes, can be used to identify and/or quantify various targets on one or more nucleic acids of interest, and may also reduce the number of spots or arrays necessary to query large sizes and/or numbers of nucleic acid molecules of interest, or fragments of those sequences.

The nucleic acid of interest may be any suitable nucleic acid molecule, for example, a genome or a chromosome, or a portion thereof, an artificial sequence, or the like. The nucleic acid may arise from, for instance, one or more chromosomes, genomic DNA, mitochondrial DNA, cDNA, RNA, mRNA or the like. Genomes, nucleic acid molecules, nucleotides, etc. are discussed in more detail below.

Arrays of spots involving nucleic acid probes are used in some embodiments of the invention. For instance, the array may contain one or more types of compound probes (in the same or in different spots on the array), and/or the array may contain multiple probes per spot (which probes may include compound probes, in some cases). The arrays of the invention can take a variety of forms. For example, an array may include a plurality of spots, at least some of which spots comprise a composition of nucleotide sequences (which may be homogeneous in some cases), and at least some of which compositions may comprise at least a first and a second oligonucleotide probe (e.g., in a compound probe). The first and second oligonucleotide probes may comprise first and second nucleotide sequences, respectively, capable of hybridizing to a first and second target nucleotide sequence in the nucleic acid molecule of interest. Further discussion of the arrays is shown below.

In some cases, the first and second nucleotide sequences of the first and second oligonucleotide probes together are not genomically contiguous when hybridized to any single strand in the nucleic acid molecule of interest. Additionally and/or alternatively, in some embodiments, the first and second nucleotide sequences of the first and second oligonucleotide probes, along with any linker segments that may be present on the first and/or second probes, together are not genomically contiguous when hybridized to any single strand in the nucleic acid molecule of interest, as described in greater detail below. In some embodiments, the first and second probes may be separated by at least 5 base pairs if hybridized to a single strand in the nucleic acid molecule of interest. In some cases, the molecules may be fragmented and/or separated, e.g., using deterministic methods. In other cases, the first and second nucleotide sequences of the first and second oligonucleotide probes may overlap if hybridized to a single strand in the nucleic acid molecule of interest. In still other cases, the first and second nucleotide sequences may arise from different nucleic acid molecules of interest, or different fragments thereof, for example, from different chromosomes, different genomes, etc.

In some embodiments, spots including at least first and second probes may involve arranging the first and second probes vertically or sequentially with respect to each other, e.g. vertically as represented in the drawings. For example, the first probe may be positioned on top of the second probe (e.g., with its 3′ end adjacent to the 5′ end of the first probe, or vice versa), or the second probe may be positioned on top of the first probe in the spot. In other instances, however, the first and second probes may be unattached to each other in the spot. As an example, the first probe may be attached directly to the surface and the second probe may be printed or synthesized on top of the first probe. Printing may include, in certain instances, chemical attachment of a first probe to a second, and/or the synthesis of one probe on top of a second probe, for instance, one or more bases at a time. The first and second probes may be chemically associated with one another on the spot (e.g., by hydrogen bonding, van der Waals forces, etc.). The height of the probes in these spots can provide another dimension for performing hybridization assays, in some cases. In another embodiment, a first probe may be positioned on top of a second probe and the first and second probes may be attached (e.g., by a covalent bond) to form a compound probe, as discussed in more detail below. As such, the present invention includes, in certain embodiments, a vertically differential array (in addition to horizontally differential array aspects, all in the context of a horizontal assay support surface where used), e.g., in order to S decrease the number of arrays or spots required in an assay for a given amount of information determinable by the array.

In some embodiments, at least first and second oligonucleotide probes may be printed on top of each other to form a single spot of an array, and the first and second probes may be capable of hybridizing to target nucleic acid sequences in a sample. In this arrangement, the first and second probes may not be chemically attached to each other, but instead can be individually and separately immobilized with respect to the array supporting surface. Other suitable arrangements of the first and second oligonucleotide probes are also possible (e.g., as discussed herein), and are contemplated within the scope of the present invention. For instance, some or all of the compound probes can be suspended in a liquid phase mixture, and then attached to a surface during hybridization, e.g., using a specific linker sequence that attaches the compound probes to predetermined sites on the surface of a substrate.

In one aspect, first and second oligonucleotide probes may be attached to one another as a single probe, forming a compound probe. A compound probe is nucleic acid comprising a nucleotide sequence comprising at least first and second probes, including first and second nucleotide sequences capable of hybridizing to first and second target nucleotide sequences, respectively, in a nucleic acid molecule of interest. The first and second nucleotide sequences may be contiguous with each other or separated from each other by a linker segment on the compound probe. In some cases, the first and second nucleotide sequences, or first and second nucleotide sequences including the linker segment, together are not genomically contiguous when hybridized to any single strand in the nucleic acid molecule of interest.

The first nucleotide sequence within the compound probe that is selected to hybridize at least a portion of the target nucleic acid may have a length of at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 125 nucleotides, at least 150 nucleotides, or at least 180 nucleotides. In some cases, the first nucleotide sequence is generally complementary to a portion of the target nucleic acid. The second nucleotide may also have a length of at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 125 nucleotides, or at least 150 nucleotides (and the length may or may not be equal to the first nucleotide sequence). In some embodiments, the second nucleotide sequence is generally or substantially complementary to a portion of the target nucleic acid (typically, a different portion than the first nucleotide sequence, e.g., on the same target nucleic acid, and/or a different target nucleic acid). In some cases, e.g., when the first and second nucleotide sequences of the probes are substantially different, the first and second nucleotide sequences may be separated (e.g., in terms of genomic coordinates) by at least 5 base pairs if hybridized to a single strand in the nucleic acid molecule of interest. Compound probes are discussed in more detail, below.

Certain aspects of the invention are generally directed to exposing compound probes (which may be attached to an array) to one or more target nucleic acids that are labeled in some fashion. By suitably labeling the target nucleic acids, the amount of binding of each of the nucleic acids to the compound probe may be determined. If the target nucleic acid(s) are distinguishably labeled in some fashion, e.g., with suitable detection entities as discussed below, the relative amounts of binding of the nucleic acid(s) to the compound probes may be used to determining information about the nucleic acids, for instance, information that relates to the presence or absence of the targets or quantification of target sequence concentrations.

As a non-limiting example, in one set of embodiments, a precursor nucleic acid may be cleaved to produce a plurality of nucleic acids, e.g., by treatment enzymes or chemicals able to cause cleavage of a nucleic acid, e.g., at specific sites, for instance, by treatment with a restriction endonuclease or other site-specific chemical cleavage method. The precursor nucleic acid may be (or arise from), for instance, one or more chromosomes, genomic DNA, mitochondrial DNA, cDNA, RNA, mRNA or the like. Those of ordinary skill in the art will be aware of suitable techniques for cleaving a nucleic acid, including site-specific methods.

The nucleic acids may be sorted and/or separated using techniques known to those of ordinary skill in the art, for instance, gel electrophoresis, capillary electrophoresis, chromatography, high pressure liquid chromatography (HPLC), mass separation, chromosome flow separation techniques, or flow cytometry. Separation may occur on the basis of physical or chemical properties, for example, size, charge, mass, absorbance, etc., and/or ratios thereof. The nucleic acids may be separated into any number of samples, for example, 2, 3, 4, 5, 6, 7, 8, 9, or 10 or more samples. It should be noted that the nucleic acids in each sample do not all necessarily have to be identical. As non-limiting examples, a first sample may have nucleic acids having a length of less than about 50 nucleotides, and a second sample may have nucleic acids having a length of greater than about 50 nucleotides; a first sample may have nucleic acids having a length of less than about 100 nucleotides, and a second sample may have nucleic acids having a length of greater than about 100 nucleotides; etc.

Some or all of the separated nucleic acids in a sample may then be labeled with a detection entity (discussed in greater detail below), and often, with distinguishable detection entities. For example, the detection entities may be determined and distinguished on the basis of color or fluorescence. In some cases, the detection entities are enzymatically incorporable into the nucleic acid, i.e., an enzyme can be used to immobilize the detection entity relative to the nucleic acid, and in some cases, such that the detection entity becomes part of the nucleic acid sequence. Depending on the application, at least 2, 3, 4, 5, 6, 7, 8, 9, 10, etc. distinguishable detection entities may be used in an experiment, e.g., based on the number of samples that the nucleic acids have been separated into such that each sample can be distinguishably identified.

Next, the nucleic acids may be exposed to one or more compound probes, for example, which may be immobilized on the surface of an array. Optionally, the nucleic acids are mixed together prior to exposure to the compound probes (i.e., the compound probe is exposed essentially simultaneously to the nucleic acids), or the nucleic acids may not be mixed together but are instead serially exposed to the compound probe. The compound probe may be selected to have a first portion able to hybridize to a first nucleic acid (or at least a portion thereof) and a second portion able to hybridize to a second nucleic acid (or at least a portion thereof). The first nucleic acid and the second nucleic acid are generally not identical and each may arise from the precursor nucleic acid. In some cases, if more than two samples of nucleic acids are present (e.g., a first nucleic acid, a second nucleic acid, and a third nucleic acid), the compound probes may be selected to hybridize to various combinations of these nucleic acids (e.g., a compound probe may hybridize to portions of the first and second nucleic acids, to the first and third nucleic acids, to the second and third nucleic acids, etc.), and/or to all of the nucleic acids (e.g., a compound probe may hybridize to portions of the first, second, and third nucleic acids), etc.

If the first and second nucleic acids each are labeled with distinguishable respective first and second detection entities, the association of the various detection entities with respect to the compound probe may then be determined. Such information may be used, for example, to determine the relative amount or degree of hybridization of the first nucleic acid with the compound probe, and the second nucleic acid with the compound probe, which can be used to determine the relative amount or ratio of the first nucleic acid to the second nucleic acid, or the concentration and/or identity of the first nucleic acid and/or the second nucleic acid in some cases. In some cases, the ratios of the first and/or second nucleic acids may also be compared to reference or control samples. Such information can also be used, in some cases, to determine structural information about the precursor nucleic acid. For example, a precursor nucleic acid may be exposed to a restriction endonuclease having a known restriction recognition site to produce the first and second nucleic acids. Determination of the amounts or concentrations of the first or second nucleic acids may then be used, for instance, to identify one or more restriction sites within the precursor nucleic acid.

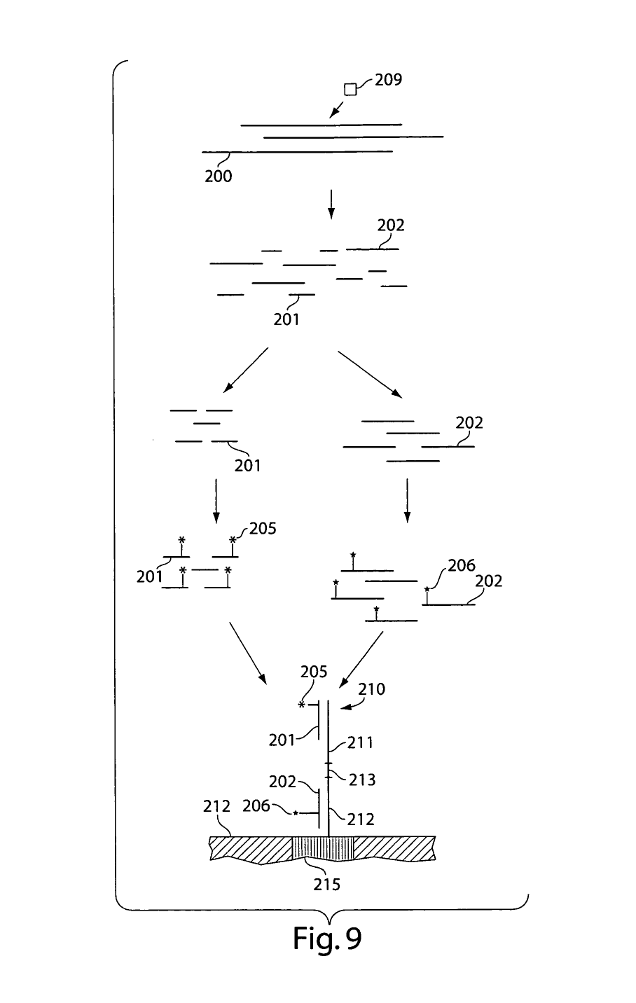

As a non-limiting example, referring now to FIG. 9, a precursor nucleic acid 200 is cleaved in some fashion, e.g., with a restriction endonuclease 209, to produce a plurality of nucleic acids, including first nucleic acid 201 and second nucleic acid 202. These nucleic acids may be separated and/or sorted, for example, based on their charge, size, hydrophobicity, etc., then labeled in some fashion. For example, nucleic acids 201 can be labeled with a first detection entity 205 in FIG. 9, while nucleic acids 202 can be labeled with second detection entities 206, which are distinguishable from first detection entities in some fashion, e.g. by color, fluorescence, energy emission, reactivity, binding affinity, etc. Next, in FIG. 9, a compound probe 210, on a surface 212 (e.g., of an array) at location 215, includes a first sequence 211 that is able to hybridize to a portion of nucleic acid 201 (but not to nucleic acid 202), and a second sequence 212 (joined through linker 213 to sequence 211, in this particular example) that is able to hybridize to a portion of nucleic acid 202. By determining association of detection entities 205 and 206 with location 215 on surface 212, the presence (i.e., hybridization) of nucleic acids 201 and 202 to compound probe 210 can be determined. In some cases, the amount of hybridization of each of nucleic acids 201 and 202 may also be compared or quantified, for example, to determine a ratio of the amount or concentration of nucleic acid 201 with respect to nucleic acid 202.

It should be noted that separation and/or sorting of a sample of different nucleic acids from a precursor nucleic acid is not necessarily required. For instance, the first and second nucleic acids may arise from separate sources (e.g., two different genomes), or the detection entities may be distinguishably applied to different nucleic acids in a sample, without requiring separation, e.g., through site-specific or sequence-specific binding.

As an example, in one set of embodiments, a precursor nucleic acid may be fragmented into a plurality of nucleic acids (e.g., using a first restriction endonuclease), then the nucleic acids end-labeled (e.g., at their 3′ and/or 5′ ends) to produce a plurality of end-labeled nucleic acids. Optionally, the end-labeled nucleic acids may be exposed to a second restriction endonuclease, which may cause cleavage of some of the end-labeled nucleic acids, e.g., into nucleic acids only labeled at one end. The end-labeled nucleic acids may then be exposed to a compound probe, as previously discussed.

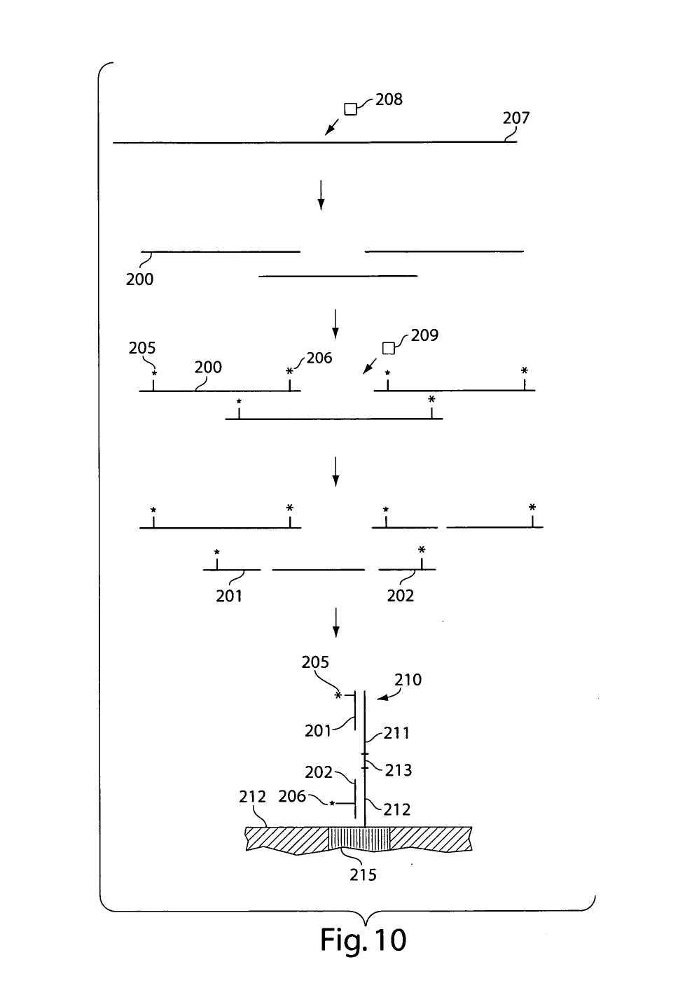

Referring now to FIG. 10, as a non-limiting example of this, a genome or other nucleic acid 207 may be cleaved in some fashion, e.g., with a restriction endonuclease 208, to produce a plurality of nucleic acids 200, which are then end-labeled with detection entities 205 and 206. The plurality of nucleic acids 200 are subsequently cleaved in some fashion, e.g., with a restriction endonuclease 208, to produce a plurality of nucleic acids, including first nucleic acid 201 and second nucleic acid 202. The nucleic acids can then be exposed to a compound probe 210, on a surface 212 (e.g., of an array) at location 215, which includes a first sequence 211 that is able to hybridize to a portion of nucleic acid 201 (but not to nucleic acid 202), and a second sequence 212 (joined through linker 213 to sequence 211) that is able to hybridize to a portion of nucleic acid 202. By determining association of detection entities 205 and 206 with location 215 on surface 212, the presence (i.e., hybridization) of nucleic acids 201 and 202 to compound probe 210 can be determined. In some cases, the amount of hybridization of each of nucleic acids 201 and 202 may also be compared or quantified, or information about the cleavage of nucleic acids 207 or 200 by restriction endonucleases 208 or 209 may be determined (e.g., determining one or more cleavage sites).

In another set of embodiments, a sample may be analyzed that contains one or more fragments or rearranged chromosome, e.g., to map the structure of the chromosome and/or of chromosomal rearrangements, for example, including translocations, such as balanced translocations (i.e., a translocation in which the chromosomes involved in the translocation are not substantially altered in size after the translocation event). Two or more chromosomes may be labeled and cleaved or fragmented (in any suitable order, as previously described) such that at least some of the nucleic acid fragments from each of the chromosomes are distinguishably labeled. Techniques for isolating or labeling a chromosome (e.g., specifically) are known to those of ordinary skill in the art, for example, Giemsa (G) banding, M banding, or spectral karyotyping (SKY). Another technique has been disclosed in a U.S. patent application filed Apr. 7, 2006, entitled “High Resolution Chromosomal Mapping,” by Barrett, et al., incorporated herein by reference.

Next, the fragments may be exposed to one or more compound probes, for example, immobilized with respect to an array. The compound probes may be selected such that a plurality of the compound probes includes a first sequence able to hybridize to the first chromosome and a second sequence able to hybridize to the second chromosome. In some cases, the first sequence of the compound probe is selected such that it is able to only hybridize the first chromosome but not the second chromosome (or any other chromosomes that may be present, in some cases), and the second sequence is selected such that it is able to only hybridize the second chromosome but not the first chromosome (or any other chromosomes that may be present). Additionally, if multiple compound probes are present, the compound probes may have different sequences that each are able to hybridize to different portions of a chromosome (i.e., a first compound probe may have a sequence able to bind a first portion of a chromosome, while a second compound probe may have a sequence able to bind a second portion of the same chromosome).

If no translocations occurred, then each of the compound probes will have a first detection entity and a second detection entity immobilized relative thereto after exposure to the chromosomal fragments. However, if a translocation event occurred, one or more of the compound probes may show immobilization of two of the same detection entity (and none of the other detection entity) instead. Since the locations on each chromosome of each of the sequences of the compound probes specifically hybridizes against are generally known, then the position of the translocation can also be determined or approximated.

As mentioned, multiple chromosomes can be simultaneously determined, by choosing appropriate compound probes. For instance, the compound probes may be selected to hybridize to various combinations of various chromosomes (e.g., a compound probe may hybridize to portions of first and second chromosomes, to first and third chromosomes, to second and third chromosomes, etc.), and/or to some or all of the chromosomes (e.g., a compound probe may hybridize to portions of the first, second, and third chromosomes, etc.).

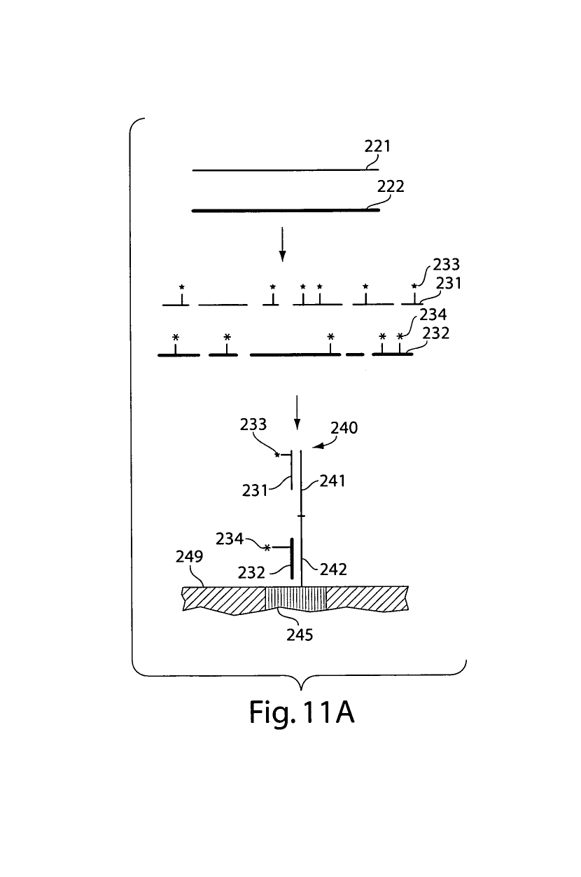

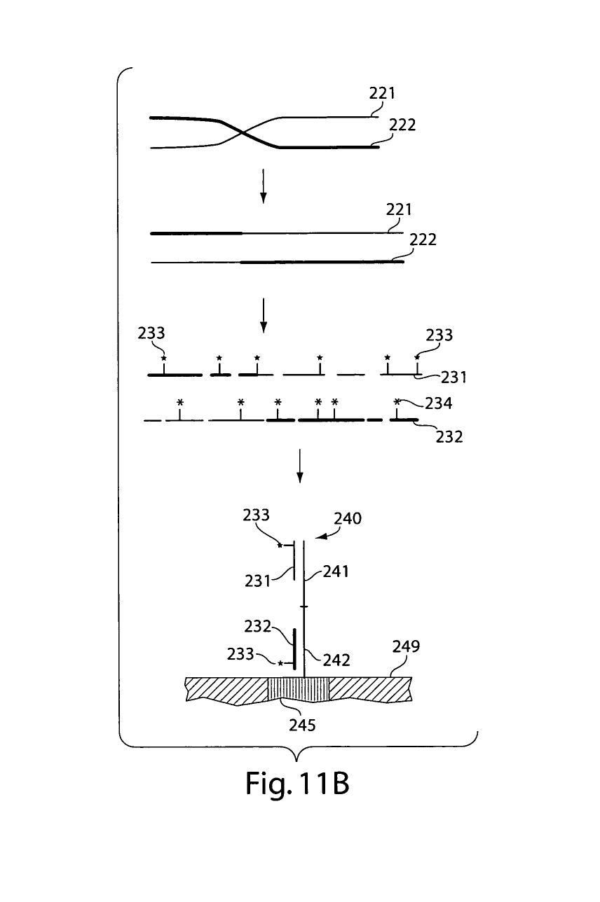

A non-limiting example is shown in FIGS. 11A and 11B. In FIG. 11A, chromosomes 221 and 222 are shown. Each of chromosomes 221 and 222 are distinguishably labeled and fragmented to produce nucleic acids 231 (with detection entities 233) corresponding to chromosome 221, and nucleic acids 232 (with detection entities 234) corresponding to chromosome 222. The nucleic acids are then exposed to a compound probe 240, on a surface 249 (e.g., of an array) at location 245, which includes a first sequence 241 that is able to hybridize to a portion of nucleic acid 231 (but not to nucleic acid 232), and a second sequence 242 that is able to hybridize to a portion of nucleic acid 232. The association of detection entities 233 and 234 with location 245 on surface 249 is then determined. Typically, both detection entities 233 and 234, corresponding to each of chromosomes 221 and 222, will be associated with location 245.

However, in FIG. 11B, a translocation event has occurred between chromosomes 221 and 222. When chromosomes 221 and 222 are fragmented and distinguishably labeled with detection entities 233 (which associate with native chromosome 221) and 234 (which associate with native chromosome 222), some of the detection entities will become immobilized relative to the other chromosome instead. After exposure to compound probe 240, on a surface 249 (e.g., of an array) at location 245, the compound probe may become associated with two detection entities 233, with no detection entities 234 present. Upon determining association of detection entities 233 and 234 with location 245 on surface 249, the lack of detection entities 234 may be indicative of a translocation event. Subsequent analysis, e.g., by comparing other compound probes used in the same assay may be used to determine the location of the translocation event.

As used herein, a “detection entity” is an entity that is capable of indicating its existence in a particular sample or at a particular location. One non-limiting example of a detection entity is a fluorescent moiety. Detection entities of the invention can be those that are identifiable by the unaided human eye, those that may be invisible in isolation but may be detectable by the unaided human eye if in sufficient quantity, entities that absorb or emit electromagnetic radiation at a level or within a wavelength range such that they can be readily detected visibly (unaided or with a microscope including a fluorescence microscope or an electron microscope, or the like), spectroscopically, or the like. Non-limiting examples include fluorescent moieties (including phosphorescent moieties), radioactive moieties, electron-dense moieties, dyes, chemiluminescent entities, electrochemiluminescent entities, enzyme-linked signaling moieties, etc. In some cases, the detection entity itself is not directly determined, but instead interacts with a second entity (a “signaling entity”) in order to effect determination (e.g., a primary antibody that recognizes the detection entity, and a labeled secondary antibody that recognizes the primary antibody). Thus, for example, coupling of the signaling entity to the detection entity may result in a determinable signal. The detection entity may be covalently attached to the oligonucleotide as a separate entity (e.g., a fluorescent molecule), or the detection entity may be integrated within the nucleic acid, for example, covalently or as an intercalation entity, as a detectable sequence of nucleotides within the oligonucleotide, etc. In some cases, the detection entity (or at least a portion thereof) forms part of the primary structure of the oligonucleotide. For instance, a number of different nucleic acid labeling protocols are known in the art and may be employed to produce a population of labeled oligonucleotides. The particular protocol may include the use of labeled primers, labeled nucleotides, nucleic acid analogs, modified nucleotides that can be conjugated with different dyes, one or more amplification steps, etc.

A variety of different detection entities may be employed, for example, fluorescent entities, isotopic entities, enzymatic entities, particulate entities, etc, as described above. Any combination of entities, e.g. first and second entities, first, second and third entities, etc., may be employed for various embodiments. Examples of distinguishable detection entities are well known in the art and include: two or more different emission wavelength fluorescent dyes, like Cy3 and Cy5, or Alexa 542 and Bodipy 630/650; two or more isotopes with different energy of emission, like ³²P and ³³P; labels which generate signals under different treatment conditions, like temperature, pH, treatment by additional chemical agents, etc.; and detection entities which generate signals at different time points after treatment. Using one or more enzymes for signal generation allows for the use of an even greater variety of distinguishable detection entities based on different substrate specificity of enzymes (e.g. alkaline phosphatase/peroxidase).

The compound probe may be of any suitable length. For instance, the compound probe may be greater than 60 nucleotides (i.e., a “long” probe), greater than 80 nucleotides, greater than 100 nucleotides, or greater than 150 nucleotides. In certain embodiments, the oligonucleotide may have a length no greater than 200 nucleotides. For example, the length of the oligonucleotide may be between 60 nucleotides and 200 nucleotides (inclusive), between 80 nucleotides and 200 nucleotides, between 90 nucleotides and 200 nucleotides, between 100 nucleotides and 200 nucleotides, between 110 nucleotides and 200 nucleotides, between 125 nucleotides and 200 nucleotides, between 150 nucleotides and 200 nucleotides, etc.

Compound probes having such nucleotide lengths may be prepared using any suitable method, for example, using de novo DNA synthesis techniques known to those of ordinary skill in the art, such as solid-phase DNA synthesis techniques, or U.S. patent application Ser. No. 11/234,701, filed Sep. 23, 2005, entitled “Methods for In Situ Generation of Nucleic Acid Molecules,” incorporated herein by reference. In some embodiments, the compound probe is immobilized with respect to a surface of a substrate. For instance, the compound probe may be immobilized at the 3′ end of the compound probe, with the 5′ end of the compound probe being furthest away from the surface of the substrate. The compound probes may be present on the surface at any suitable density (or present in a spot or feature on the surface of the substrate, e.g., in an array, as discussed below), for example, at a density of at least about 0.01 pmol/mm², at least about 0.03 pmol/mm², at least about 0.1 pmol/mm², at least about 0.3 pmol/mm², at least about 1 pmol/mm², etc. In one instance, the density of compound probes on the solid support is between about 0.01 pmol/mm² and about 1 pmol/mm². In some cases, while compound probes at different spots or locations on a surface may not be identical, compound probes at a spot may be substantially identical, e.g., at least about 25%, at least about 50%, or at least about 75%, or at least about 95% of the compound probes at a feature may comprise an identical sequence composition and length. In certain embodiments, some or all of the compound probes on the array are substantially homogenous or highly uniform in terms of compound probe composition. Advantageously, background noise and non-selective signal are reduced in the hybridization signal.

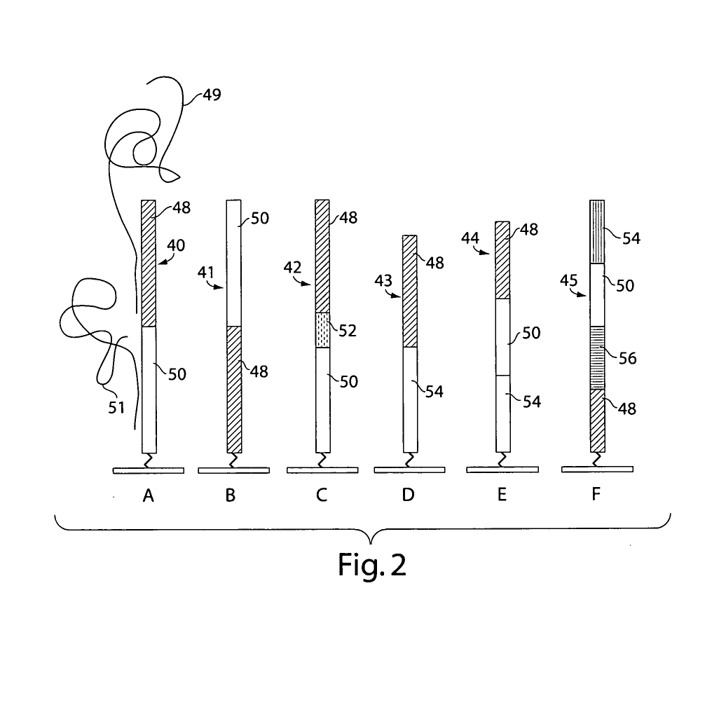

Configurations and arrangements of probes within a compound probe may vary, as illustrated in more detail below. Each probe of a compound probe may have a suitable length such that it can be used to hybridize to target nucleotide sequences in a biological sample. As shown in FIG. 2A, compound probe 40 includes at least a first probe 48 and a second probe 50. First probe 48 and second probe 50 may be made up of different nucleic acid sequences and may hybridize to different portions of a nucleic acid molecule of interest, or different nucleic acid molecules. For instance, all, or a portion, of probe 48 may hybridize to a first target nucleotide sequence indicated as strand 49 in the figure, and all, or a portion, of probe 50 may hybridize to a portion of target nucleotide sequence 51.

In other instances, a compound probe may include at least first and second probes that are substantially similar. For instance, all, or portions, of the nucleotide sequences of the first and second probe may comprise the same sequence. For example, the first and second probes may be designed to hybridize to an essentially identical portion of a nucleic acid molecule of interest. In such a case, the first and second probes may have the same lengths in some embodiments; however, in other embodiments, the first and second probes may have different lengths. A compound probe including first and second probes that are substantially similar may be advantageous for increasing the accuracy of hybridization in an assay.

As compound probes may vary, an array or array set of the invention can include one, or a combination, of types of compound probes described herein. Arrays and array sets of probes and compound probes are described in more detail below. In addition, an array or array set may comprise any combination of both compound probes and typical non-compound (e.g., regular) probes.

As illustrated in FIGS. 2A and 2B, the orientation of probes 48 and 50 may vary on compound probe 40 compared to compound probe 41. Certain designs of compound probes, e.g., orientations of probes on a compound probe and/or ordering of the compound probes within the probe, may be advantageous when considering, for example, decreasing the noise of a signal and/or the ability to synthesize the probe. Design considerations for compound probes are described in more detail below.

FIGS. 2A and 2B show oligonucleotide probes that are contiguous with each other on the compound probe. For instance, the first probe comprising a first nucleotide sequence may be directly adjacent to the second probe comprising a second nucleotide sequence. In other cases, the first and second nucleotide sequences of first and second oligonucleotide probes, respectively, are not contiguous with each other on the compound probe. For example, compound probes may be separated by a linker segment 52, which may comprise specific nucleic acid sequences (FIG. 2C). In some embodiments, for example, the specific nucleic acid sequence of linker segment 52 does not include a sequence that makes probes 48 and 50, along with linker segment 52, genomically contiguous when each of the probes and segments is hybridized to any single strand in the nucleic acid molecule of interest, as discussed in more detail below. As shown in FIG. 2C, probes 48 and 50 may be shorter (i.e., include few nucleotide sequences) if linker segments are included on the compound probe (e.g., compared to the lengths probes 48 and 50 in FIG. 2A and 2B). However, in other instances, e.g., depending on the lengths of the probes and/or the total length of the compound probe, the lengths of probes 48 and 50 may not differ compared to compound probes without linker segments. In some embodiments, e.g., as illustrated in FIG. 2D, compound probe 43 may include probe 48 and a probe 54 having a sequence that is at least substantially complementary to the sequence of probe 48.

A compound probe may optionally comprise a third probe 54, as shown in compound probe 44 of FIG. 2E, or a fourth probe 56, as shown in compound probe 45 of FIG. 2F. Of course, greater than four probes, e.g., five, six, seven, or higher numbers of probes, can be included on a compound probe. I.e., in some cases, a compound probe can comprise greater than 2, greater than 4, greater than 6, greater than 8, greater than 10, greater than 12, greater than 14, or greater than 16 probes. As noted, at least two probes of a compound probe may have different sequences and may hybridize to a particular portion of the nucleic acid molecule of interest, i.e., greater than 50%, greater than 70%, greater than 90%, or about 100% of the sequences of a first probe may differ from those of a second probe, as described in more detail below.

Compound probes 40-45 of FIG. 2 may have various lengths and/or may comprise various numbers of nucleotides. For instance, a compound probe may comprise greater than or equal to 20 nucleotides, greater than or equal to 40 nucleotides, or greater than or equal to 60 nucleotides. In some cases, compound probe 40 (and/or compound probes 41-45) forms a long, high quality oligonucleotide. E.g., the compound probe may comprise greater than or equal to 80 nucleotides, greater than or equal to 100 nucleotides, greater than or equal to 120 nucleotides, greater than or equal to 140 nucleotides, or greater than or equal to 160 nucleotides. In certain instances, compound probe 40 (and/or compound probes 41-45) may be a 60-mer, 70-mer, 90-mer, 110-mer, 130-mer, 150-mer, or 170-mer.

A compound probe may include a first oligonucleotide probe comprising a first nucleotide sequence capable of hybridizing to a first target nucleotide sequence in a nucleic acid molecule of interest and a second oligonucleotide probe comprising a second nucleotide sequence capable of hybridizing to a second target nucleotide sequence in the nucleic acid molecule of interest. The degree of hybridization of a nucleotide sequence (e.g., the first nucleotide sequence) to a target nucleotide sequence (e.g., the first target nucleotide sequence) can depend on the particular application and/or hybridization conditions. For instance, in some cases, a nucleotide sequence that hybridizes to a target nucleotide sequence in a nucleic acid molecule of interest may include 100% matched nucleotide pairs (e.g., 100% of the nucleotide sequence of the oligonucleotide probe may hybridize with the target nucleotide sequence). In other cases, a nucleotide sequence that is capable of hybridizing to a target nucleotide sequence may include greater than 95%, greater than 90%, greater than 80%, greater than 70%, greater than 60% matched nucleotide pairs, greater than 40% matched nucleotide pairs, or greater than 20% matched nucleotide pairs. In certain embodiments, the degree of hybridization between a nucleotide sequence (e.g., of an oligonucleotide probe) and a target nucleotide sequence means that these sequences are capable of hybridizing under certain conditions, e.g., under stringent conditions or array assay conditions, i.e., to produce a detectable signal.

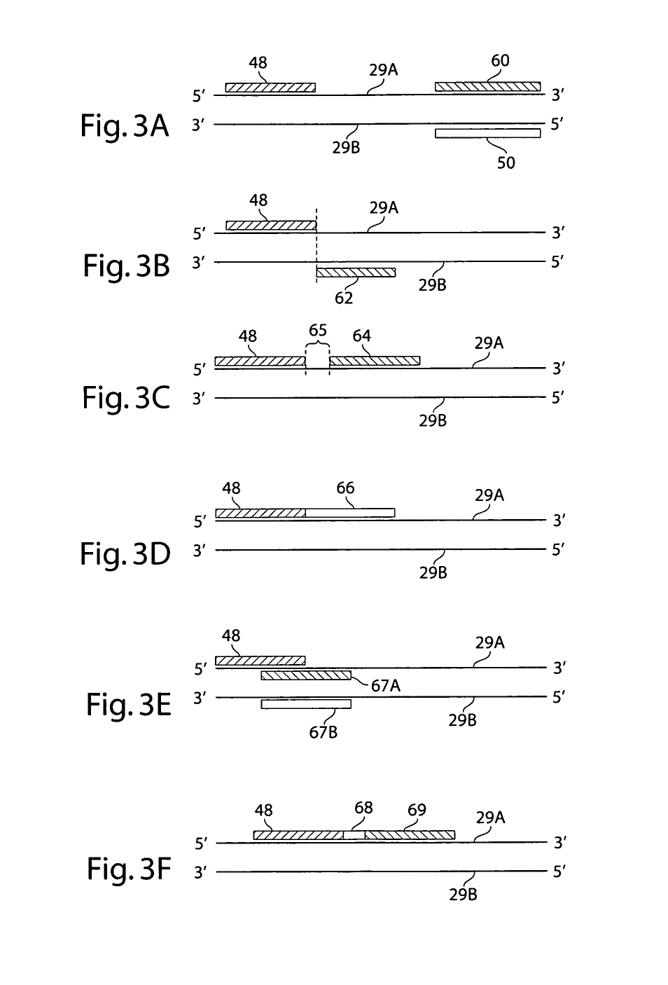

In one embodiment, a compound probe includes at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest, wherein the first and second nucleotide sequences of the first and second oligonucleotide probes, respectively, may be contiguous with each other on the compound probe or separated from each other by a linker segment on the compound probe, and wherein the first and second nucleotide sequences or first and second nucleotide sequences including the linker segment, together are not genomically contiguous when hybridized to any single strand in the nucleic acid molecule of interest. In primary embodiments, the first and second nucleotides sequences of the first and second oligonucleotide probes, respectively, together are not genomically contiguous when hybridized to any single strand in the nucleic acid molecule of interest, e.g., if the first and second probes are contiguous on the compound probe. Referring now to both FIGS. 2 and 3, where FIG. 3 illustrates various arrangements of oligonucleotide probes hybridized to target sequences, compound probe 40 of FIG. 2A (and/or compound probes 41 of FIG. 2B) may include probes 48 and 50 that are not genomically contiguous when hybridized to strands 29A or 29B of FIG. 3A. In another embodiment, a compound probe may comprise probes 48 and 60, which are not contiguous on strand 29A of the nucleic acid molecule of interest. In yet another embodiment, as shown in FIG. 3B, a compound probe may include probes 48 and 62 that are also not genomically contiguous on any single strand in the nucleic acid molecule of interest.

In some cases where first and second oligonucleotide probes of a compound probe hybridize to a single strand in the nucleic acid molecule of interest (or hybridize to complementary strands of those regions of interest, this arrangement included as an embodiment), the nucleotide sequences of the first and second probes are separated by a number of bases, for example, at least 1 base, at least 2 bases, at least 5 bases, or at least 10 bases, when hybridized to the single strand. For instance, as shown in FIG. 3C, probes 48 and 64, which may be combined to form a compound probe, may be separated by spacing 65. Spacing 65 may be at least 1 base, at least 2 bases, at least 5 bases, or at least 10 bases long on strand 29A. As shown in FIG. 3D, a probe represented by probes 48 and 66, which are contiguous when hybridized to strand 29A, does not define a compound probe according to some embodiments (e.g., when probes 48 and 66 are contiguous on a single probe), since the individual probes are contiguous when hybridized to the strand.

In some cases, the first and second nucleotide sequences of first and second oligonucleotide probes of a compound probe can overlap if hybridized to a single strand (or a complementary strand) in the nucleic acid molecule of interest. For instance, as shown in FIG. 3E, a compound probe may include probes 48 and 67A, which overlap with each other if each of the probes are hybridized to strand 29A. In another embodiment, a compound probe may include probes 48 and 67B, which overlap if each of the probes are hybridized to complementary strands in the nucleic acid molecule of interest.

In other embodiments, the first and second nucleotide sequences of the first and second oligonucleotide probes of a compound probe, respectively, together can be genomically contiguous when hybridized to any single strand in the nucleic acid molecule of interest, if the first and second sequences of the compound probe are separated by a particular linker segment. For instance, a compound probe can include probes 48 and 66 of FIG. 3D if probes 48 and 66 are not contiguous on the compound probe, e.g., if they are present in compound probe 42 of FIG. 2C as probes 48 and 50. In other embodiments, the first and second nucleotide sequences of the first and second oligonucleotide probes of a compound probe, along with any linker segments that may be present on the compound probe, together are not genomically contiguous when hybridized to any single strand in the nucleic acid molecule of interest. For example, as shown in FIG. 3F, a probe including probes 48, segment 68, and probe 69, in that consecutive order as shown in FIG. 3F (and without any additional linker segments), does not make up a compound probe. However, an embodiment comprising probe 69, segment 68, and probe 48 (e.g., where the 3′ end of probe 69 is connected to the 5′ end of segment 68, and the 3′ end of segment 68 is connected to the 5′ end of probe 48) can comprise a compound probe.

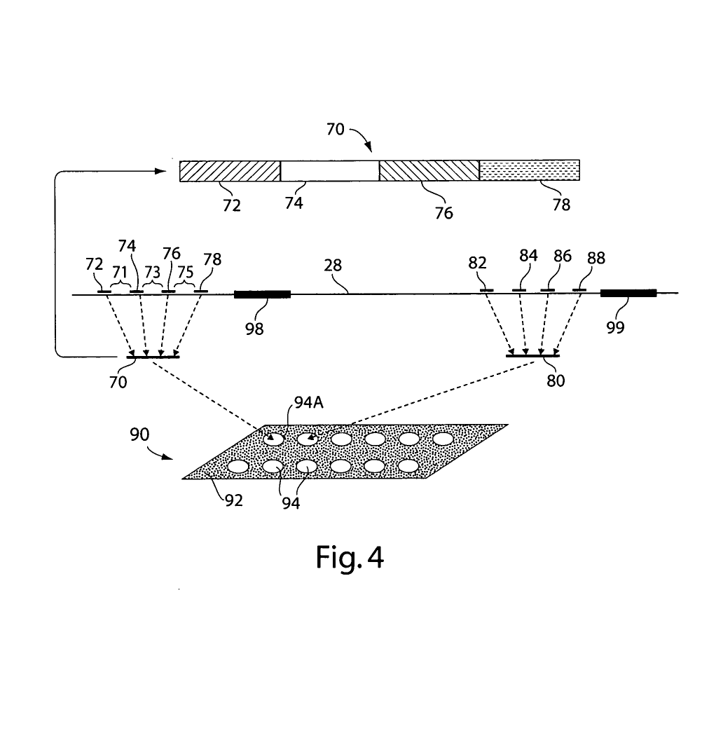

In the embodiment illustrated in FIG. 4, compound probe 70 comprises a series of probes 72, 74, 76, and 78, which can be designed to hybridize to nucleotide sequences located on different parts of a nucleic acid molecule of interest 28. As illustrated in this particular embodiment, the target nucleotide sequences that can hybridize to probes 72, 74, 76, and 78 are not contiguous with each other on the nucleic acid molecule of interest, since they are separated by sections 71, 73, and 75 of the nucleic acid molecule of interest. In one embodiment, sections 100, 73, and 75 each comprise greater than 5 bases. Probes may be separated by a relatively small number of bases (e.g., less than 50 bases) in cases where higher resolution assays are desired. In other cases, sections 71, 73, and 75 may comprise higher numbers of bases (e.g., greater than 100 bases), e.g., when it is desirable to include probes that span nucleic acid molecules of interest having relatively large numbers of bases. As such, the length of sections 71, 73, and 75 can vary depending on the particular application. For example, the average distance between two consecutive probes hybridized to a nucleic acid molecule of interest may be between 0-10 bases, between 1-50 bases, between 50-100 bases, between 100-300 bases, between 300-500 bases, between 500-1000 bases, between 1-10 kb, or greater than 10 kb.

In some cases, the spacing between consecutive probes that are hybridized to a nucleic acid molecule of interest may be substantially equivalent (e.g., consecutive probes may be separated by about 300 bases). In other cases, the spacing between consecutive probes may differ along particular portions of the nucleic acid molecule of interest. For example, if it is known that a biological phenomena is associated with a particular portion of the nucleic acid, that portion may include a higher resolution of probes than a portion that is not associated with the biological phenomena.

As illustrated in the embodiment shown in FIG. 4, compound probes 70 and 80 may be immobilized on, e.g., covalently attached to, locations on solid support 92 (e.g., a substrate surface). Each distinct compound probe (e.g., compound probes 70 and 80) on the support may be present as a homogeneous composition and concentration of multiple copies of the probe on the substrate surface, e.g., as spots 94 on the surface of the substrate. A series of probes 72, 74, 76, and 78 that make up compound probe 70 are adjacent to each other along nucleic acid molecule of interest 28, and are genomic neighbors because they are on, or near, one particular gene (e.g., gene 98). A probe that is a genomic neighbor of another probe may be said to be on, or near, the same gene in a nucleic acid molecule of interest. In some cases, the nearness or proximity of a first and a second probe relative to one another may be defined at least in part by a certain number of bases. For instance, a first probe near a second probe may be separated by less than about 107 bases, less than about 10⁶ bases, than about 10⁵ bases, than about 104 bases, less than about 1,000 bases, less than about 500 bases, less than about 300 bases, or less than about 100 bases. In another embodiment, the nearness or proximity of a first and a second probe may be defined at least in part by whether or not they are part of the same gene on the nucleic acid molecule of interest. For example, a first and a second probe that are on the same gene may be genomic neighbors and may be said to be near one another, while probes that are on different genes in the nucleic acid molecule of interest are not genomic neighbors and are not near one another.

In other embodiments, a compound probe may include probes that are not on, or near, the same gene in the nucleic acid molecule of interest. For example, assays may be designed to include compound probes made up of probes that are not located on the same gene in the 20 nucleic acid molecule of interest. For example, in one embodiment, a compound probe may include a first probe on, or near, gene 98 (e.g., one of probes 72, 74, 76, or 78), and a second probe on, or near, gene 99 (e.g., one of probes 82, 84, 86, or 88). In other cases, the compound probe does not include two probes on, or near, gene 98, or two probes on, or near, gene 99. As described in more detail below, such factors are important considerations for designing arrays and for deconvoluting signals obtained from hybridization.

In some cases, more than one oligonucleotide probe, such as a compound probe, may be present. For instance, in certain embodiments, at least 100, at least 1,000, at least 10,000, or at least 100,000 non-identical oligonucleotide probes are present, e.g., on spots within an array. A spot of an array can include a homogeneous composition of at least first and second oligonucleotide probes that may be unattached, or attached as a single probe (e.g., a compound probe), according to another aspect of the invention. Arrays are discussed in greater detail, below. The plurality of compound probes may be present on a surface of the same solid support. The compound probes may be immobilized on, e.g., covalently attached to, different and, in certain embodiments, known, locations on the a solid support (e.g., substrate surface). In certain embodiments, each distinct compound probe nucleotide sequence of the support is typically present as a composition of multiple copies of the compound probe on the substrate surface, e.g., as a spot or feature on the surface of the substrate. The number of distinct nucleic acid sequences, and hence spots or similar structures, present on the array may vary, but is generally at least 2, usually at least 5 and more usually at least 10, where the number of spots on the array may be as a high as 50, 100, 500, 1000, 10,000 or higher, depending on the intended use of the array. The spots of distinct nucleotide sequences present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. However, in some cases, the distinct nucleotide sequences may be unpatterned or comprise a random pattern.

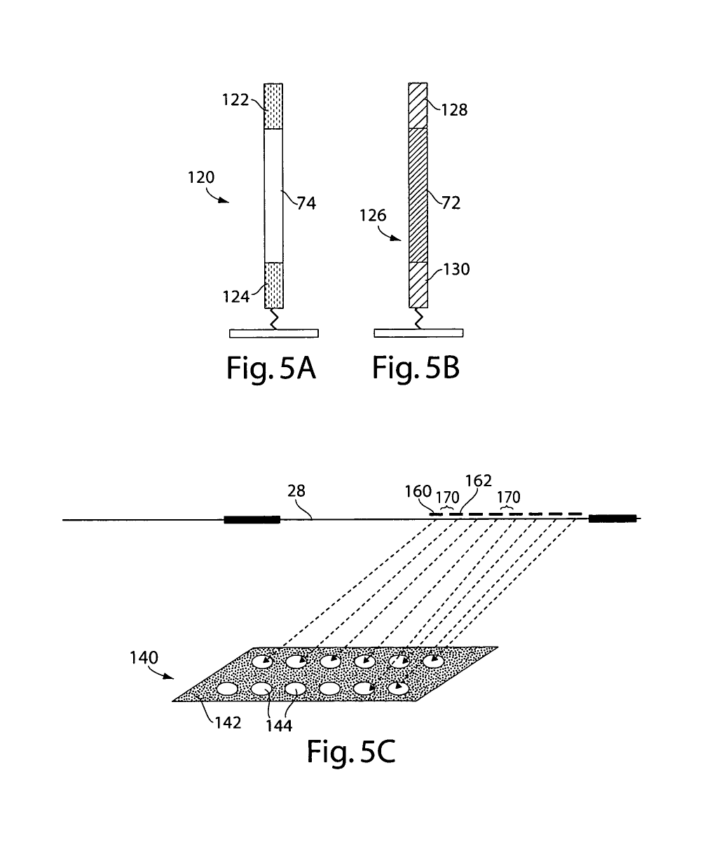

A variety of methods can be used to deconvolute the signals attained from hybridization on a array or array sets, including signals from spots comprising more than one probe on each spot. In one embodiment, after performing an initial set of assays using array 90, the general areas of interest showing hybridization (i.e., signals or hits) can be deconvoluted by performing a second round of hybridization. This second assay can be designed to tailor the results of the first set of assays and only the hit areas of the first assay can be included. For example, during a first set of assays, if spot 94A of FIG. 4 comprising compound probe 70 produced a signal after hybridization, probes 72, 74, 76 and 78 of compound probe 70 may be included as individual spots in a second assay involving array 140 of FIG. 5C. As shown in the embodiment illustrated in FIG. 5, full length probes can be used in array 140. For instance, probe 74, being a 40mer in compound probe 70 in FIG. 4, may be included in array 140 as a 60-mer, including regions 122 and 124 that flank probe 74. Regions 122 and 124 may be chosen at least in part by the sequence of the nucleic acid molecule of interest. For example, when probe 74 is hybridized to the nucleic acid molecule of interest, regions 122 and 124 may also hybridize to the nucleic acid molecule of interest in their positions flanking probe 74. Similarly, probe 72, which was a 40mer on compound probe 70 of FIG. 4, may be included in array 140 as full length probe 126 including probe 72, as well as regions 128 and 130 that flank probe 72. Regions 128 and 130 may also be chosen at least in part by the sequence of the nucleic acid molecule of interest. Of course, probe 72 may be flanked with only one region 128 or 130. Alternatively, probe 72 may be used as is on array 140, e.g., without flanking regions. The length of probes 120 and 126 can vary, e.g., depending on the assay and/or the hybridization conditions desired. Probes in array 140 may have a length of, for example, greater than 20 nucleotides, greater than 40 nucleotides, greater than 60 nucleotides, greater than 80 nucleotides, or greater than 100 nucleotides.

As illustrated in the embodiment of FIG. 5C, array 140 includes a higher resolution of probes (e.g., a smaller distance between probes on nucleic acid molecule of interest 28) compared to the probes used in array 90 (FIG. 4C). For instance, sections 170 may separate adjacent probes such as probes 160 and 162, and these sections may each comprise fewer numbers of base pairs that those separating adjacent probes in the first assay involving array 90. E.g., sections 170 may comprise less than about 300 bases, e.g., from about 1-50 bases, from about 50-200 bases, or from about 100-300 bases.

Since spots 144 each comprise a homogenous composition of a single probe, the signals produced or detected after hybridization of the probes and target nucleotides sequences can enable determination of which probe of compound probe 70 gave rise to the spot signal of array 90 of FIG. 4. In some cases, the probes of array 140 can be chosen from the compound probes that gave the strongest signals in array 90, e.g., the probes of the top 10%, top 20%, top 30%, or top 50% of the compound probes that gave the strongest signals may be included in array 140. As such, a single spot of array 140 may allow determination of the location of a biological phenomenon in terms of chromosomal coordinates in the nucleic acid molecule of interest. In some instances, in order to verify a signal from a spot, a series of signals from the spots may be correlated. In other cases, a series of spots may be required to determine the location of a biological phenomenon in terms of chromosomal coordinates in the nucleic acid molecule of interest.

Additional assay arrangements and deconvolution techniques, as well as other compound probes, are described in a U.S. patent application filed on even date herewith, entitled “Compound Probes and Methods of Increasing the Effective Probe Densities of Arrays,” by Leproust, et al., a U.S. patent application filed on even date herewith, entitled “Methods of Increasing the Effective Probe Densities of Arrays,” by Gordon, et al., and a U.S. patent application filed on even date herewith, entitled “Analysis of Arrays,” by Gordon, et al., each incorporated herein by reference.

The arrays may be contacted with a sample under conditions that permit hybridization between target nucleotide sequences of the sample and sequences of the oligonucleotide probes. After hybridization and scanning, one or more spots may fluoresce to produce spot signals. In some cases, it may be desirable to determine which probe contributed to the spot signal (e.g., to determine which of the probes of the compound probe the target nucleotide sequence was hybridized). In other cases, however, it is not necessary to determine which probe contributed to the spot signal in order to determine the location of a biological phenomena in terms of chromosomal coordinates in the nucleic acid molecule of interest. In some embodiments, the signal from one spot may be correlated to the signal from one or more other spots in order to determine the location of the biological phenomena.

In one embodiment, the constraints of having probes that are non-genomic neighbors of one another on the same compound probe can aid in the deconvolution of signals obtained upon hybridization. In some cases, knowledge of the expected correlation between neighboring probes can also help in deconvoluting the contribution of each probe of a compound probe from a spot signal.

In some cases, the signal associated with a biological phenomena at a specific location on a nucleic acid molecule of interest is distributed to probes that are genomic neighbors. For instance, since fragmentation of the nucleic acid of interest is performed randomly, fragments including different nucleotide sequences may include the same signal associated with the biological phenomena. When the fragment length exceeds the probe spacing (in genomic coordinates), a biological phenomena can generate a signal that is spread across a set of probes in a genomic region. For example, if the median fragment length is about 800 bp and the average probe spacing is about 30 bp, then a given biological phenomena can contribute a signal across a genomic “neighborhood” of about 26 probes (e.g., 800 bp divided by 30 bp spacing). Some of the embodiments presented here use this expected correlation among probes that are genomic neighbors for the deconvolution of signals from compound probes.

In one embodiment, deconvolution of signals obtained upon hybridization may be performed at least in part by the fragment distribution, which can be generally approximated (e.g., about 800 bp fragments for a typical ChiP-chip sonication protocol) or inferred (e.g., from precise measurement of individual samples via gel electrophoresis or a Bio-Analyzer). Deconvolution can be achieved by analyzing a spot signal of compound probes in the genomic context of the probes making up the compound probes. For example, if a particular compound probe including a first and a second probe produces a spot signal, then it can be determined which probe of the compound probe is/are responsible for the signal by looking at the spot signal in the context of the signals of the other compound probes comprising the genomic neighbors of the first probe, and then repeating for the second probe, and so on. The analysis of an expected distribution can take on many forms, e.g., ranging from peak-fitting (e.g., of intensities and/or ratios) to a more comprehensive error model that takes into account the error in the probe intensities and/or knowledge of the expected signal distribution. Such an error model can propagate these errors to make a final estimate of the confidence in identifying signal-producing regions.

In addition to the methods described above, methods that increase the ability to resolve underlying biological events as well as overall signal-to-noise performance, through design of the compound probes, are now described. One potential problem associated with compound probes is homology noise at the probe boundaries. For instance, the nucleotide sequences that span concatenation points at the probe boundaries may be unintentionally homologous with other parts of the genome, creating additional biological noise, and leading to non-informational, spurious hybridization on assays. In one embodiment, a method to reduce boundary homology noise in a compound probe includes the use of linker segments between probes. Linker segments, as shown in embodiment 52 of FIG. 2C, may be carefully selected for each adjacent pair of probes within a compound probe to minimize homology noise. For instance, for a compound probe including first and second probes having first and second nucleotide sequences, respectively, a boundary region created by the first and second nucleotide sequences and the linker segment may produces less noise than a boundary region created by the first and second nucleotide sequences without the linker segment, when hybridized to target nucleotides sequences of a biological sample.

Typically, linker segments are short sequences added between two probes of a compound probe. These segments may be, for example, less than 20 pb, less than 10 bp, less than 6 bp, or less than 4 bp in length. However, in other embodiments, longer linker segments may be used. Linker segments may have a variable length, e.g., within a compound probe or between compound probes. In one embodiment, the length and/or sequences of linker segments are randomly selected and/or randomly assigned to compound probes. In another embodiment, the length and/or sequences of linker segments can be selected based on a pre-computed database of linker segments with good homology scores, which indicate low homology noise. For instance, the database of linker segments may be derived at least in part by genomes of other organisms. Or, the database of linker segments may be derived at least in part by sections of the nucleic acid molecule of interest that are known to have good homology scores. For instance, sequences that are known to not show up frequently in the nucleic acid molecule of interest may be suitable linker segments for use in some compound probes.

In one embodiment, a method of assigning at least a first probe and a second probe to a compound probe includes identifying the boundaries between the first and second probes. The amount of homology noise between the probe boundaries (e.g., of the first and second probes) and a particular sequence and a nucleic acid molecule of interest may be analyzed. If the noise between the probe boundaries and sequences of the nucleic acid molecule of interest is low, the linker segment between the first and second probes may not be required. However, if the noise is high, a suitable linker segment may be positioned between and first and second probes in the compound probe. As described above, a database of linker segments may identify the unique sequence that is suitable for the insertion between the first and second probes in order to decrease the amount of homology noise. Of course, the boundary region between first and second probes can differ depending on the order of the first and second probes on the compound probe. For instance, as shown in FIGS. 2A and 2B, the order of probes on a compound probe can differ. As part of the analysis of identifying suitable boundaries between probes of the compound probe, the noise contribution of each arrangement of probes in a compound probe can be evaluated. As such, the arrangement of probes that gives boundary regions having the lowest amount of noise between regions of the nucleic acid molecule of interest may be chosen.

In another embodiment, a method of assigning at least a first probe and a second probe to a compound probe includes choosing probes that have a low probability of self-hybridization, e.., to avoid the formation of hairpins on the spot. However, in certain embodiments, compound probes including probes that can self-hybridized may be useful as controls. In such embodiments, a compound probe may include a first nucleotide sequence and a second nucleotide sequence, wherein the second nucleotide sequence is the complement of the first nucleotide sequence.

In another embodiment, the arrangement (e.g., ordering) of the probes within the compound probe may be selected to minimize boundary homology noise. This can be done by evaluating at least two, several, or all possible arrangements (and/or a subset of possible arrangements) of probes within a compound probe, and selecting the arrangement expected to have the overall lowest boundary homology noise. In addition, this method can be used in conjunction with the linker method presented previously. For instance, in one embodiment, a method of designing a compound probe comprises selecting candidate probes for a compound probe, the candidate probes comprising at least a first oligonucleotide probe comprising a first nucleotide sequence capable of hybridizing to a first target nucleotide sequence in a nucleic acid molecule of interest, and at least a second oligonucleotide probe comprising a second nucleotide sequence capable of hybridizing to a second target nucleotide sequence in the nucleic acid molecule of interest. The method can involve estimating the boundary homology noise of at least two possible arrangements of the first and second oligonucleotide probes within a compound probe, and selecting the arrangement estimated to have the overall lowest boundary homology noise. In some cases, the boundary homology noise of all possible arrangements of the first and second oligonucleotide probes within a compound probe can be estimated, and the arrangement estimated to have the overall lowest boundary homology noise can be selected.

In cases in which compound probes with linker segments are desired, a method of designing a compound probe may further comprise selecting a linker segment from a database of linker segments. The boundary homology noise of at least two possible arrangements (or in some cases, all possible arrangements) of the first and second oligonucleotide probes together with the linker segment within a compound probe may be estimated, and the arrangement estimated to have the overall lowest boundary homology noise can be selected. The database of linker segments can be derived at least in part by sections of the nucleic acid molecule of interest that are known to have good homology scores and/or at least in part by sections of a genome that is different from that of the nucleic acid molecule of interest (e.g., the genome of another organism). Additionally, in some cases, large numbers of candidate linker sequences may be generated and screened against a background of whole genome sequences, e.g., to ensure minimal binding, for instance, in a CGH assay.

In some embodiments, the methods described above may use a mechanism to evaluate boundary homology noise. This can be done by using existing sequence matching tools such as BLAST, BLAT, and/or MegaBLAST. The system can exclude the expected genome matches from the probes of a compound probe, and use any remaining matches to assess boundary homology noise. However, in some cases, this method could be very computationally expensive, e.g., for large genomes.

A method that can be more efficient (though in some cases, perhaps less precise) may include simply looking for exact matches of some given length (k) created at probe boundary regions (e.g., with or without a linker segment). This can be done by pre-computing a hash/lookup table of all unique k-length segments for a given genome. To evaluate a concatenation point, a k-size window can move one base pair at a time across the boundary point and each sequence may be looked up in the table to estimate homology noise. The overall boundary noise estimate for the compound probe can include a combination of the noise estimates for each boundary within the compound probe.

In another embodiment, ability to resolve underlying biological events can be controlled by taking advantage of information about expected correlation among probes to allocate probes to compound probes. A simple example is as follows: for assays (such as ChIP-Chip assays) where the genomic DNA is fragmented, one can expect genomically adjacent probes, sufficiently close together, to show highly correlated signals. In general, a set of probes with expected correlated signals can be spread out among different compound probes, such that there is only one probe of the set in a given compound probe. Other assays may have other correlations which can be leveraged to increase resolving power and/or to control a particular method of deconvoluting signals from hybridization.

For instance, in another embodiment, if it known that a first and second region of a nucleic acid molecule of interest have a high likelihood of being associated with a biological phenomenon, probes within the first and second regions are not put together in a single compound probe. After this constraint, probes that combine to form a compound probe may be chosen from random positions along the nucleic acid molecule of interest. As such, a compound probe may include only one probe representative of a binding site for a biological phenomenon. Consequently, it may be possible to take a description of an assay and put different design parameters to best allocate probes to compound probes and/or compound probes to a particular arrangement on an array in order to tailor the arrangement of probes and compound probes to a particular assay. Other constraints of assigning probes to compound probes and/or the assignment of compounds to particular spots on an array or array set may allow other associations between signals that can used to increase resolution and/or decrease the number of spots per array.

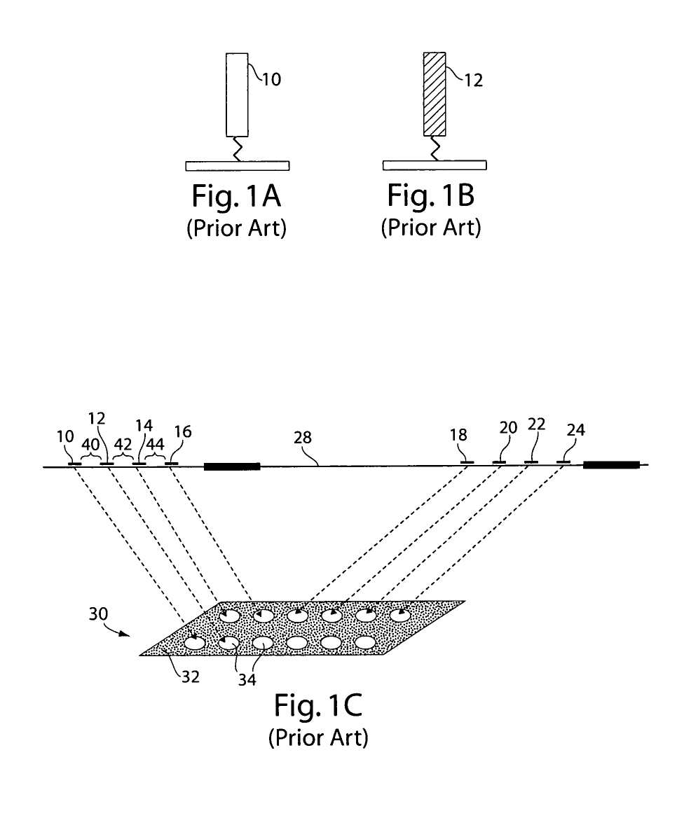

The spots comprising multiple probes per spot (e.g., compound probes) and arrays of the invention find may use in a variety of different applications, including analyte detection applications in which the presence of a particular analyte in a given sample is detected (e.g., qualitatively or quantitatively). Articles and methods of the invention involving spots comprising multiple probes per spot (e.g., compound probes) can be used in any suitable application that uses typical probe arrays such as those shown in FIG. 1. Examples of specific applications include, but are not limited to, array CGH, location analysis (ChIP-Chip), gene synthesis, mutation detection, probe synthesis, aptamer synthesis, therapeutics, microRNA analysis, methylation analysis, amplification methods and the like. Those of ordinary skill in the art may know protocols for carrying out such assays.

Generally, in detection methods relying on oligonucleotides attached to an array, the sample suspected of comprising a target nucleic acid molecule of interest can be contacted with an array under conditions sufficient for the target nucleic acid molecule to hybridize to its respective binding pair member that is present on the array. Thus, if the target nucleic acid molecule of interest is present in the sample, it can hybridize to the array at the site of its binding partner and a complex may be formed on the array surface. The presence of this hybridized complex on the array surface can then be detected, e.g., through use of a signal production system, e.g., an isotopic or fluorescent label present on the target nucleic acid molecule, etc. The presence of the target nucleic acid molecule in the sample can then be deduced from the detection of hybridized complexes on the substrate surface in combination with the methods described herein.

Another aspect of the invention is generally directed to a kit. A “kit,” as used herein, typically defines a package including one or more of the compositions of the invention, and/or other compositions associated with the invention, for example, one or more nucleic acid probes as previously described. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, modifying, assembling, storing, packaging, preparing, mixing, diluting, and/or preserving the compositions components for a particular use.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, assembly, storage, packaging, and/or preparation of the compositions and/or other compositions associated with the kit. In some cases, the instructions may also include instructions, for example, for a particular use. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

The kits may also comprise containers, each with one or more of the various reagents and/or compositions. The kits may also include a collection of immobilized oligonucleotide targets, e.g., one or more arrays of targets, and reagents employed in genomic template and/or labeled probe production, e.g., a highly processive polymerase, exonuclease resistant primers, random primers, buffers, the appropriate nucleotide triphosphates (e.g. dATP, dCTP, dGTP, dTTP), DNA polymerase, labeling reagents, e.g., labeled nucleotides, and the like. Where the kits are specifically designed for use in CGH applications, the kits may further include labeling reagents for making two or more collections of distinguishably labeled nucleic acids according to the subject methods, an array of target nucleic acids, hybridization solution, etc.

The following documents are incorporated herein by reference: U.S. patent application Ser. No. 10/448,298, filed May 28, 2003, entitled “Comparative Genomic Hybridiztion Assays using Immobilized Oligonucleotide Targets with Initially Small Sample Sizes and Compositions for Practicing the Same,” by M. T. Barrett, et al., published as U.S. Patent Application Publication No. 2004/0241658 on Dec. 2, 2004; and International Patent Application No. PCT/US2003/041047, filed Dec. 22, 2003, entitled “Comparative Genomic Hybridization Assays using Immobilized Oligonucleotide Features and Compositions for Practicing the Same,” by L. K. Bruhn, et al., published as WO 2004/058945 A2 on Jul. 15, 2004. The following documents are also incorporated herein by reference: a U.S. patent application filed on even date herewith, entitled “Compound Probes and Methods of Increasing the Effective Probe Density of Arrays,” by Leproust, et al.; a U.S. patent application filed on even date herewith, entitled “Methods of Increasing the Effective Probe Density of Arrays,” by Gordon, et al.; and a U.S. patent application filed on even date herewith entitled “Analysis of Arrays,” by Gordon, et al.

Unless defined otherwise, all technical and scientific terms used herein have the same meaning as commonly understood by one of ordinary skill in the art to which this invention belongs. Still, certain terms are defined below for the sake of clarity and ease of reference. Interspersed with these definitions is additional disclosure of various embodiments of the invention.

The term “sample,” as used herein, relates to a material or mixture of materials, typically, although not necessarily, in fluid form, containing one or more components of interest. The term “biological sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

When two items are “associated” with one another, they are provided in such a way that it is apparent one is related to the other such as where one references the other. For example, an array identifier can be associated with an array by being on the array assembly (such as on the substrate or a housing) that carries the array or on or in a package or kit carrying the array assembly.

“Stably attached” or “stably associated with” means an item's position remains substantially constant.

“Contacting” means to bring or put together. As such, a first item is contacted with a second item when the two items are brought or put together, e.g., by touching them to each other.

“Depositing” means to position, place an item at a location, or otherwise cause an item to be so positioned or placed at a location. Depositing includes contacting one item with another. Depositing may be manual or automatic, e.g., “depositing” an item at a location may be accomplished by automated robotic devices.

The term “biomolecule” means any organic or biochemical molecule, group or species of interest that may be formed in an array on a substrate surface. Non-limiting examples of biomolecules include peptides, proteins, amino acids, and nucleic acids.

A “biopolymer” is a polymeric biomolecule comprising one or more types of repeating units. Biopolymers are typically found in biological systems and particularly include polysaccharides (e.g., carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source. For example, a “biopolymer” may include DNA (including cDNA), RNA, oligonucleotides, and PNA and other polynucleotides as described in U.S. Pat. No. 5,948,902, incorporated herein by reference. A “biomonomer” refers to a single unit, which can be linked with the same or other biomonomers to form a biopolymer (e.g., a single amino acid or nucleotide with two linking groups, one or both of which may have removable protecting groups). A biomonomer fluid or biopolymer fluid references a liquid containing either a biomonomer or biopolymer, respectively (typically in solution).

The term “peptide,” as used herein, refers to any compound produced by amide formation between an alpha-carboxyl group of one amino acid and an alpha-amino group of another group. The term “oligopeptide,” as used herein, refers to peptides with fewer than about 10 to 20 residues, i.e., amino acid monomeric units. As used herein, the term “polypeptide” refers to peptides with more than 10 to 20 residues. The term “protein,” as used herein, refers to polypeptides of specific sequence of more than about 50 residues.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, epsilon-nicotinoyl-lysine, isonipecotic acid, tetrahydroisoquinoleic acid, alpha acid, sarcosine, citrulline, cysteic acid, t-butylglycine, t-butylalanine, phenylglycine, cyclohexylalanine, beta-alanine, 4-aminobutyric acid, and the like.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art, synthesis of nucleic acids of this type may utilize, in some cases, an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include, but are non limited to, deoxyribonucleotides (DNA), ribonucleotides (RNA), or other polynucleotides which are C-glycosides of a purine or pyrimidine base. The oligomer may be defined by, for example, about 2-500 monomers, about 10-500 monomers, or about 50-250 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “X-mer” refers to an oligonucleotide that has a defined length, which is usually a sequence of at least 3 nucleotides, in some cases, 4 to 14 nucleotides, in other cases 5 to 20, 5 to 30, 8 to 50, 8 to 60, 50 to 100, 50 to 120, 50 to 150, 100-200 nucleotides in length, or longer. For instance, a 60-mer refers to an oligonucleotide having a sequence of 60 nucleotides.

The,term “X-mer precursors,” sometimes referred to as “oligonucleotide precursors” refers to a nucleic acid sequence that is complementary to a portion of the target nucleic acid sequence. The oligonucleotide precursors are sequences of nucleoside monomers joined by phosphorus linkages (e.g., phosphodiester, alkyl and aryl-phosphate, phosphorothioate, phosphotriester), or non-phosphorus linkages (e.g., peptide, sulfamate and others). They may be natural or non-natural (e.g., synthetic) molecules of single-stranded DNA and single-stranded RNA with circular, branched or linear shapes, and optionally including domains capable of forming stable secondary structures (e.g., stem-and-loop and loop-stem-loop structures). The oligonucleotide precursors contain a 3′-end and a 5′-end.

The term “complementary, “complement,” or “complementary nucleic acid sequence” refers to the nucleic acid strand that is related to the base sequence in another nucleic acid strand by the Watson-Crick base-pairing rules. In general, two sequences are complementary when the sequence of one can hybridize to the sequence of the other in an anti-parallel sense wherein the 3′-end of each sequence hybridizes to the 5′-end of the other sequence and each A, T(U), G, and C of one sequence is then aligned with a T(U), A, C, and G, respectively, of the other sequence. RNA sequences can also include complementary G/U or U/G base pairs.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA,” as used herein, refer to a polymer comprising ribonucleotides. The terms “deoxyribonucleic acid” and “DNA,” as used herein, mean a polymer comprising deoxyribonucleotides. The term “oligonucleotide” as used herein denotes single stranded nucleotide multimers of from about 10 to 200 nucleotides and up to about 500 nucleotides in length. For instance, the oligonucleotide may be greater than about 60 nucleotides, greater than about 100 nucleotides or greater than about 150 nucleotides. The term “mRNA” means messenger RNA.

As used herein, a “target nucleic acid sample” or a “target nucleic acid” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

The term “target nucleic acid sequence” refers to a sequence of nucleotides to be identified, detected, or otherwise analyzed, usually existing within a portion or all of a polynucleotide. In the present invention, the identity of the target nucleotide sample or sequence may or may not be known. The identity of the target nucleotide sequence may be known to an extent sufficient to allow preparation of various sequences hybridizable with the target nucleotide sequence and of oligonucleotides, such as probes and primers, and other molecules necessary for conducting methods in accordance with the present invention and so forth. Determining the sequence of the target nucleic acid includes in its definition, determining the sequence of the target nucleic acid or sequences within regions of the target nucleic acid to determine the sequence de novo, to resequence, and/or to detect mutations and/or polymorphisms. In some cases, target nucleic acid sequences are present in a biological sample of interest.

The terms “target nucleic acid” and “nucleic acid molecule of interest” are used interchangeably herein. A target nucleic acid or a nucleic acid molecule of interest may represent, for example, a genome (e.g., a “target genome”) or a transcriptome (e.g., a “target transcriptome”).

The target sequence may contain from about 30 to 5,000 or more nucleotides, or from 50 to 1,000 nucleotides. In some cases, the target nucleotide sequence is generally a fraction of a larger molecule. In other cases, the target nucleotide sequence may be substantially the entire molecule, such as a polynucleotide as described above. The minimum number of nucleotides in the target nucleotide sequence is selected to assure that the presence of a target polynucleotide in a sample is a specific indicator for the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g., transcription of a DNA template into RNA) and the efficiency of identification, detection, amplification, and/or other analysis of the target nucleotide sequence, where appropriate.

As used herein, a “reference nucleic acid sample” or a “reference nucleic acid” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, alkylated riboses, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “polynucleotide” or “nucleic acid” refers to a polymer composed of nucleotides, natural compounds such as deoxyribonucleotides or ribonucleotides, or compounds produced synthetically (e.g., PNA as described in U.S. Pat. No. 5,948,902 and the references cited therein), which can hybridize with naturally-occurring nucleic acids in a sequence specific manner analogous to that of two naturally occurring nucleic acids, e.g., can participate in Watson-Crick base pairing interactions. The polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. Isolation of a polynucleotide from the natural state often results in fragmentation. It may be useful to fragment longer target nucleic acid sequences, particularly RNA, prior to hybridization to reduce competing intramolecular structures.

The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fumgi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, cDNA, and the like.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method.

For purposes of this invention, the polynucleotide, or a cleaved fragment obtained from the polynucleotide, will usually be at least partially denatured or single stranded or treated to render it denatured or single stranded. Such treatments are well known in the art and include, for instance, heat or alkali treatment, or enzymatic digestion of one strand. For example, dsDNA can be heated at 90 to 100 degrees Celsius for a period of about 1 to 10 minutes to produce denatured material.

The nucleic acids may be generated by in vitro replication and/or amplification methods such as the Polymerase Chain Reaction (PCR), asymmetric PCR, the Ligase Chain Reaction (LCR) and so forth. The nucleic acids may be either single-stranded or double-stranded. Single-stranded nucleic acids are preferred because they lack complementary strands that compete for the oligonucleotide precursors during the hybridization step of the method of the invention.

The term “oligonucleotide” refers to a polynucleotide, usually single stranded, usually a synthetic polynucleotide but may be a naturally occurring polynucleotide. The length of an oligonucleotide is generally governed by the particular role thereof, such as, for example, probes (e.g., compound probes), primers, X-mers, and the like. Various techniques can be employed for preparing an oligonucleotide. Such oligonucleotides can be obtained by biological synthesis or by chemical synthesis. For short oligonucleotides (i.e., up to about 100 nucleotides), chemical synthesis will frequently be more economical as compared to the biological synthesis. In addition to economy, chemical synthesis provides a convenient way of incorporating low molecular weight compounds and/or modified bases during specific synthesis steps. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. Methods of oligonucleotide synthesis include phosphotriester and phosphodiester methods (Narang, et al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al. (Proc. Nat. Acad. Sci. USA 91:5022-5026, 1994). In some cases, synthesis of certain oligonucleotides (e.g., compound probes) can be performed according to methods disclosed in U.S. Patent Publication No. 2005/0214779, filed Mar. 29, 2004, entitled “Methods for in situ generation of nucleic acid arrays,” which is incorporated herein by reference.

Generally, as used herein, the terms “oligonucleotide” and “polynucleotide” are used interchangeably. Further, generally, the term “nucleic acid molecule” also encompasses oligonucleotides and polynucleotides.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×19 base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements, and amplification of any subchromosomal region or DNA sequence. In certain embodiments, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

The “genomic source” is the source of the initial nucleic acids from which the nucleic acid probes are produced, e.g., as a template in the labeled nucleic acid protocols described in greater detail herein. The genomic source may be prepared using any convenient protocol. In some embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in certain embodiments, genomic DNA representing the entire genome from a particular organism, tissue or cell type. A given initial genomic source may be prepared from a subject, for example a plant or an animal that is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 MB, such that the may be about 1 Mb or smaller, e.g., less than about 500 kb, etc.

If a surface-bound nucleic acid or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array elements, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

The terms “hybridize” or “hybridization,” as is known to those of ordinary skill in the art, refer to the specific binding or duplexing of a nucleic acid molecule to a particular nucleotide sequence under suitable conditions, e.g., under stringent conditions. For example, under stringent conditions, hybridized duplexes comprising an DNA oligonucleotide probe and its corresponding DNA target sequence may form double-stranded DNA duplexes which, in large part, is formed of Watson-Crick base pairs. The terms “hybridization,” and “hybridizing,” in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the hybridization of the two sequences. Increased stringency can be achieved by elevating the temperature, increasing the ratio of co-solvents, lowering the salt concentration, and the like. Hybridization also includes in its definition the transient hybridization of two complementary sequences. It is understood by those skilled in the art that non-covalent hybridization between two molecules, including nucleic acids, obeys the laws of mass action. Therefore, for purposes of the present invention, hybridization between two nucleotide sequences for a length of time that permits primer extension and/or ligation is within the scope of the invention. The term “hybrid” refers to a double-stranded nucleic acid molecule formed by hydrogen bonding between complementary nucleotides.

The term “stringent conditions” (or “stringent hybridization conditions”) as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent conditions are the summation or combination (totality) of both hybridization and wash conditions.

In certain embodiments, an array is contacted with a nucleic acid sample under stringent assay conditions, i.e., conditions that are compatible with producing hybridized pairs of biopolymers of sufficient affinity to provide for the desired level of specificity in the assay while being less compatible to the formation of hybridized pairs between members of insufficient affinity. Stringent assay conditions are the summation or combination (totality) of both hybridization conditions and wash conditions for removing unhybridized molecules from the array.

Stringent conditions (e.g., as in array, Southern or Northern hybridizations) may be sequence dependent, and are often different under different experimental parameters. Stringent conditions that can be used to hybridize nucleic acids include, for instance, hybridization in a buffer comprising 50% formamide, 5×SSC (salt, sodium citrate), and 1% SDS at 42° C., or hybridization in a buffer comprising 5×SSC and 1% SDS at 65° C., both with a wash of 0.2×SSC and 0.1% SDS at 65° C. Other examples of stringent conditions include a hybridization in a buffer of 40% formamide, 1 M NaCl, and 1% SDS at 37° C., and a wash in 1×SSC at 45° C. In another example, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional examples of stringent conditions include hybridization at 60° C. or higher and 3×SSC (450 mM sodium chloride/45 mM sodium citrate) or incubation at 42° C. in a solution containing 30% formamide, 1 M NaCl, 0.5% sodium lauryl sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to another nucleic acid (for example, when a nucleic acid has hybridized to a nucleic acid probe). Wash conditions used to identify nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The terms “high stringency conditions” or “highly stringent hybridization conditions,” as previously described, generally refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Additional hybridization methods are described in references describing CGH techniques (Kallioniemi et al., Science 1992;258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, e.g., Gall et al. Meth. Enzymol. 1981 ;21 :470-480 and Angerer et al., In Genetic Engineering: Principles and Methods, Setlow and Hollaender, Eds. Vol 7, pgs 43-65 (Plenum Press, New York 1985). See also U.S. Pat. Nos 6,335,167, 6,197,501, 5,830,645, and 5,665,549, the disclosures of which are herein incorporated by reference.

The term “oligonucleotide probe” or “probe” refers to an oligonucleotide employed to hybridize to a portion of a polynucleotide such as another oligonucleotide or a target nucleotide sequence. The design and preparation of the oligonucleotide probes are generally dependent upon the sequence to which they hybridize. Oligonucleotide probes can include natural or non-natural nucleotides.

“Addressable sets of probes” and analogous terms refer to the multiple known regions of different moieties of known characteristics (e.g., base sequence composition) supported by or intended to be supported by a solid support, i.e., such that each location is associated with a moiety of a known characteristic and such that properties of a target moiety can be determined based on the location on the solid support surface to which the target moiety hybridizes under stringent conditions.

The phrases “nucleic acid molecule bound to a surface of a solid support,” “probe bound to a solid support,” “probe immobilized with respect to a surface,” “target bound to a solid support,” or “polynucleotide bound to a solid support” (and similar terms) generally refer to a nucleic acid molecule (e.g., an oligonucleotide or polynucleotide) or a mimetic thereof (e.g., comprising at least one PNA, UNA, and/or LNA monomer) that is immobilized on the surface of a solid substrate, where the substrate can have a variety of configurations, e.g., including, but not limited to, planar substrates, non-planar substrate, a sheet, bead, particle, slide, wafer, web, fiber, tube, capillary, microfluidic channel or reservoir, or other structure. The solid support may be porous or non-porous. In certain embodiments, collections of nucleic acid molecules are present on a surface of the same support, e.g., in the form of an array, which can include at least about two nucleic acid molecules. The two or more nucleic acid molecules may be identical or comprise a different nucleotide base composition. As used herein, the terms “bound to a solid support” and “attached to a solid support” may be used interchangeably unless context dictates otherwise.

A solid support, in some embodiments, is non-porous. In certain embodiments, a non-porous support comprises a bead. As used herein, a “non-porous support” refers to a support having a pore size that essentially excludes synthesis reagents (e.g., such as biopolymer precursors or solutions for preparing biopolymers, including but not limited to deblocking and purging solutions) from entering the support (e.g., penetrating the surface). In one aspect, to the extent there are any openings/pores in a surface of a support, the openings/pores can be less than about 100 Angstroms, less than about 60 angstroms, less than about 50 Angstroms, less than about 25 Angstroms, etc. Included in this definition are supports having these specified size restrictions or properties in their natural state or which have been treated to reduce the size of any openings/pores to obtain these restrictions/properties. In certain embodiments, supports include non-porous beads. Such beads can be fabricated as is known in the art, for example, as described in U.S. Patent Publication No. 2003/0225261.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. The term “feature” is used interchangeably herein, in this context, with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

In the broadest sense, the arrays of many embodiments are arrays of polymeric binding (or hybridization) agents, where the polymeric binding agents may be any one or more of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). In some cases, the arrays are arrays of polypeptides, e.g., proteins or fragments thereof.

An “array” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain.

An “array set” includes one or more arrays tailored to a particular assay. An array set may include more than one array, e.g., when there are too many spots or features to fit on a single substrate and/or spots are spread over multiple substrates. The multiple substrates may be said to be part of an array set. An example of an array set includes a “10-set” product, which is on ten glass slides with about 440,000 spots (e.g., about 44k spots per slide). An “array” and “array set” may be used interchangeably herein in some embodiments of the invention.

The term “substrate” as used herein refers to a surface upon which marker molecules or probes, e.g., an array, may be adhered. Glass slides are the most common substrate for biochips, although fused silica, silicon, plastic, and other materials are also suitable. The substrate may be formed in essentially any shape. In one set of embodiments, the substrate has at least one surface which is substantially planar. However, in other embodiments, the substrate may also include indentations, protuberances, steps, ridges, terraces, or the like. The substrate may be formed from any suitable material, depending upon the application. For example, the substrate may be a silicon-based chip or a glass slide. Other suitable substrate materials for the arrays of the present invention include, but are not limited to, glasses, ceramics, plastics, metals, alloys, carbon, agarose, silica, quartz, cellulose, polyacrylamide, polyamide, polyimide, and gelatin, as well as other polymer supports or other solid-material supports. Polymers that may be used in the substrate include, but are not limited to, polystyrene, poly(tetra)fluoroethylene (PTFE), polyvinylidenedifluoride, polycarbonate, polymethylmethacrylate, polyvinylethylene, polyethyleneimine, polyoxymethylene (POM), polyvinylphenol, polylactides, polymethacrylimide (PMI), polyalkenesulfone (PAS), polypropylene, polyethylene, polyhydroxyethylmethacrylate (HEMA), polydimethylsiloxane, polyacrylamide, polyimide, various block co-polymers, etc.

Any given substrate may carry any number of oligonucleotides on a surface thereof. In some cases, one, two, three, four, or more arrays may be disposed on a surface of the substrate. Depending upon the use, any or all of the arrays may be the same or different from one another and each may contain multiple spots, or elements or features of different moieties (for example, different polynucleotide sequences). A spot or feature of an array is generally homogeneous in composition and in concentration. A region at a particular predetermined location (e.g., an “address”) on the array can detect a particular target or set of targets (although a spot or feature may incidentally detect non-targets of that spot or feature in some cases). The target for which the spot or feature is specific is, in representative embodiments, known. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from about 10 micrometers to 1.0 cm. In other embodiments each feature may have a width in the range of 1.0 micrometers to 1.0 mm, 5.0 micrometers to 500 micrometers, 10 micrometers to 200 micrometers, etc. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature or interspot areas may be present in some embodiments which do not carry any oligonucleotide (or other biopolymer or chemical moiety of a type of which the features are composed). Such interfeature areas may be present where the arrays are formed by processes involving drop deposition of reagents but may not be present when, for example, light directed synthesis fabrication processes are used. It will be appreciated though, that the interfeature areas, when present, could be of various sizes and configurations. In other embodiments, however, oligonucleotides may be present in interspot areas. In one particular embodiment, spots are arranged adjacent one another such that there are no interspot areas between each spot.

The substrate may have thereon a pattern of locations (or elements) (e.g., rows and columns) or may be unpatterned or comprise a random pattern. The elements may each independently be the same or different. For example, in certain cases, at least about 25% of the elements are substantially identical (e.g., comprise the same sequence composition and length). In certain other cases, at least 50% of the elements are substantially identical, or at least about 75% of the elements are substantially identical. In certain cases, some or all of the elements are completely or at least substantially identical. For instance, if nucleic acids are immobilized on the surface of a solid substrate, at least about 25%, at least about 50%, or at least about 75% of the oligonucleotides may have the same length, and in some cases, may be substantially identical.

An “array layout” or “array characteristics” refers to one or more physical, chemical or biological characteristics of the array, such as positioning of some or all the features within the array and on a substrate, one or more dimensions of the spots or elements, or some indication of an identity or function (for example, chemical or biological) of a moiety at a given location, or how the array should be handled (for example, conditions under which the array is exposed to a sample, or array reading specifications or controls following sample exposure).

Each array may cover an area of less than 100 cm², or even less than 50 cm², 10 cm², 1 cm², 0.5 cm², or 0.1 cm² In certain embodiments, the substrate carrying the one or more arrays will be shaped as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. In some cases, the array will have a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. In some instances, with arrays that are read by detecting fluorescence, the substrate may be of a material that emits low fluorescence upon illumination with the excitation light. Additionally, in some cases the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident thereon, as may be measured across the entire integrated spectrum of such illuminating light or alternatively at 532 nm or 633 nm.

In certain embodiments of particular interest, in situ prepared arrays are employed. In situ prepared oligonucleotide arrays, e.g., nucleic acid arrays, may be characterized by having surface properties of the substrate that differ significantly between the feature and interfeature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water, as known in the art and further describedin in copending application Ser. No. 10/449,838, the disclosure of which is herein incorporated by reference. Other features of in situ prepared arrays that make such array formats of particular interest in certain embodiments of the present invention include, but are not limited to: feature density, oligonucleotide density within each feature, feature uniformity, low intra-feature background, low interfeature background, e.g., due to hydrophobic interfeature regions, fidelity of oligonucleotide elements making up the individual features, array/feature reproducibility, and the like. The above benefits of in situ produced arrays assist in maintaining adequate sensitivity while operating under stringency conditions required to accommodate highly complex samples.

In certain embodiments, a nucleic acid sequence may be present as a composition of multiple copies of the nucleic acid molecule on the surface of the array, e.g., as a spot or element on the surface of the substrate. The spots may be present as a pattern, where the pattern may be in the form of organized rows and columns of spots, e.g., a grid of spots, across the substrate surface, a series of curvilinear rows across the substrate surface, e.g., a series of concentric circles or semi-circles of spots, or the like. The density of spots present on the array surface may vary, for example, at least about 10, at least about 100 spots/cm², at least about 1,000 spots/cm², or at least about 10,000 spots/cm². In other embodiments, however, the elements are not arranged in the form of distinct spots, but may be positioned on the surface such that there is substantially no space separating one element from another.

In certain aspects, in constructing arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In certain aspects, an array may be optimized for one type of genome scanning application compared to another, for example, the array can be enriched for intergenic regions compared to coding regions for a location analysis application. In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic regions (e.g., non-coding regions which exclude introns and untranslated regions, i.e, comprise non-transcribed sequences) of a nucleotide sample of interest.

In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. Int. Pat. Apl. WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning. Themed arrays also can be fabricated for use in gene expression assays, for example, to detect expression of genes involved in selected pathways of interest, or genes associated with particular diseases of interest.

In one embodiment, a plurality of probes on the array is selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In still other aspects, probes on the array comprise at least coding sequences. In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

In some embodiments, the array may be referred to as addressable. An array is “addressable” when it has multiple regions of different moieties (e.g., different nucleic acids) such that a region (i.e., an element or “spot” of the array) at a particular predetermined location (i.e., an “address”) on the array may be used to detect a particular target or class of targets (although an element may incidentally detect non-targets of that element). In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., nucleic acid molecules, to be evaluated by binding with the other).

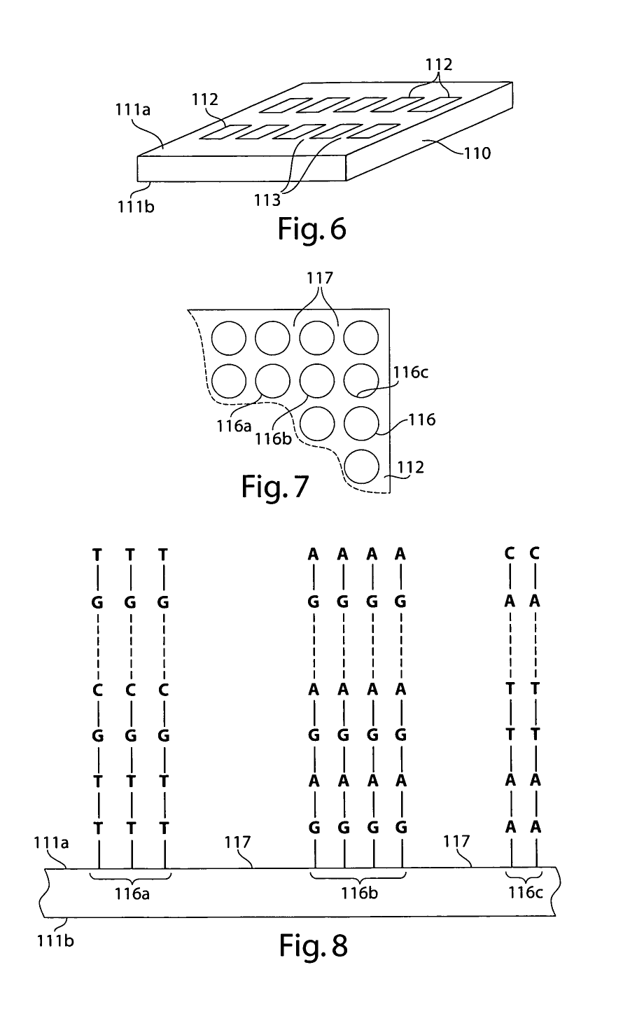

An example of an array is shown in FIGS. 6-8, where the array shown in this representative embodiment includes a contiguous planar substrate 110 carrying an array 112 disposed on a rear surface 111 b of substrate 110. It will be appreciated though, that more than one array (any of which are the same or different) may be present on rear surface 111 b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots or elements of interest, as discussed above, are found. For example, the scan region may be that portion of the total area illuminated from which resulting fluorescence is detected and recorded. For the purposes of this invention, the scan region includes the entire area of the slide scanned in each pass of the lens, between the first element of interest, and the last element of interest, even if there are intervening areas which lack elements of interest. An “array layout” refers to one or more characteristics of the features, such as element positioning on the substrate, one or more feature dimensions, and an indication of a moiety at a given location.

In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

The arrays can be fabricated using drop deposition from pulsejets of either oligonucleotide precursor units (such as monomers) in the case of in situ fabrication, or the previously obtained oligonucleotide. Such methods are described in detail in, for example, in U.S. Pat. Nos. 6,242,266, 6,232,072, 6,180,351, 6,171,797, or 6,323,043, or in U.S. patent application Ser. No. 09/302,898, by Caren et al., filed Apr. 30, 1999, and the references cited therein. These references are each incorporated herein by reference. Other drop deposition methods can be used for fabrication, as previously described herein.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for ChIP-chip analysis.” In certain aspects, a CGH array provides probes for screening or scanning a genome of an organism and comprises probes from a plurality of regions of the genome.

In using an array made by the method of the present invention, the array will be exposed in certain embodiments to a sample (for example, a fluorescently labeled target nucleic acid molecule) and the array then read. Reading of the array may be accomplished, for instance, by illuminating the array and reading the location and intensity of resulting fluorescence at various locations of the array (e.g., at each spot or element) to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 6,756,202 or 6,406,849, each incorporated herein by reference.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one set of embodiments, the control target molecules in a population are present at a level comparable to a haploid amount of a gene represented in the target population. In other embodiments, the control target molecules are present at a level comparable to a diploid amount of a gene. In still other embodiments, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain embodiments, test and reference populations of nucleic acids may be applied separately to separate but identical arrays (e.g., having identical probe molecules) and the signals from each array can be compared to determine relative copy numbers of the nucleic acids in the test and reference populations.

Arrays may also be read by any other method or apparatus than the foregoing, with other reading methods, including other optical techniques (for example, detecting chemiluminescent or electroluminescent labels) or electrical techniques (where each feature is provided with an electrode to detect hybridization at that feature in a manner disclosed in, e.g., U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition).

The term “tag” as used herein, generally refers to a chemical moiety, which is used to identify a nucleic acid sequence, and preferably but not necessarily to identify a unique nucleic acid sequence. For instance, “tags” with different molecular weights can be distinguishable by mass spectrometry, and may be used to reduce the mass ambiguity between two or more nucleic acid molecules with different nucleotide sequences, but with the identical molecular weights. The “tag” may be covalently linked to an X-mer precursor, e.g., through a cleavable linker.

As used herein, “not genomically contiguous” means that a first hybridizing segment (e.g., a first probe of a compound probe) and a second hybridizing segment (e.g., a second probe of a compound probe) are not contiguous when hybridized to a nucleic acid molecule of interests (e.g., a target genome). Non-genomically contiguous sequences may be separated by at least 5 b, at least 100 b, at least 1 kb, at least 10 kb, at least 100 kb and in certain cases may be on different chromosomes in a genome, e.g., a mammalian, e.g., human genome, etc. A “signal” is a numerical measurement or an estimated (e.g., calculated) measurement of a characteristic of a signal received from scanning an array. Thus, a signal is a numerical score that quantifies some aspect of a spot/spot signal. For example, a mean intensity value of a spot is a statistic, as is a standard deviation value for pixel intensity within a spot. A signal can also refer to the “enrichment” of the probe, including, but not limited to, so-called “one-color” measurements, ratios between channels of a “two-color” assay, difference between channels of a “two-color” assay, or variants of these measures that are adjusted by normalization or by using estimates of the error in the measurements.

As used herein, “enrichment” refers to a signal or a meaningful combination of signals (e.g., of two colors of the same spot). For instance, in some embodiments, the scanner can measure two signal strengths for each feature: (1) the strength of a signal at a first wavelength that indicates the strength of the binding between the probes of a given feature and a control target; and (2) the strength of a signal at a second wavelength that indicates the strength of the binding between the probes of the aforementioned given feature and a test target. The ratio between the two signal strengths indicates the extent by which the test target differs from the control, and may indicate that a particular region of the genome is of interest. Thus, a high ratio between signal strengths from a test target and a control target (test:control) typically indicates a region of interest. The ratio is one of a number of possible ways of measuring the “enrichment” of the test target. Others include so-called “one-color” measurements (test), difference (test-control), or variants of these measures that are adjusted by normalization or by using estimates of the error in the measurements (test-control)/error. In certain embodiments, “signal” and “enrichment” are used interchangeably herein.

A “hybridizing segment” is a region of an oligonucleotide that hybridizes with a target nucleic acid.

As used herein, “homology noise” (or “cross-hybridization noise”) refers to a signal for a probe that arises due to the hybridization of DNA fragments to it that do not correspond to the genomic location it represents. This behavior can occur, for instance, when DNA fragments from different locations in the genome have sequences similar to all, or a portion of, a probe (e.g., high homology). This behavior can also occur in some methods involving formation of compound probes, e.g., when sequences that form the hybridizing segments of the compound probe are concatenated, creating new sequences at the concatenation point.

It will also be appreciated that throughout the present application, that words such as “cover,” “base,” “front,” “back,” and “top” are used in a relative sense only. The word “above” used to describe the substrate and/or flow cell is meant with respect to the horizontal plane of the environment, e.g., the room, in which the substrate and/or flow cell is present, e.g., the ground or floor of such a room.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. All definitions, as defined and used herein, should be understood to control over dictionary definitions, definitions in documents incorporated by reference, and/or ordinary meanings of the defined terms.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. In this specification and the appended claims, the singular forms “a,” “an” and “the” include plural reference unless the context clearly dictates otherwise.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

“Optional” or “optionally,” as used herein, means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

It should also be understood that, unless clearly indicated to the contrary, in any methods claimed herein that include more than one step or act, the order of the steps or acts of the method is not necessarily limited to the order in which the steps or acts of the method are recited.

All publications mentioned herein are incorporated herein by reference for the purpose of describing and disclosing the invention components that are described in the publications that might be used in connection with the presently described invention.

In the claims, as well as in the specification above, all transitional phrases such as comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
providing an oligonucleotide probe;
exposing the oligonucleotide probe to a first nucleic acid labeled with a first detection entity such that at least a portion of the first nucleic acid hybridizes to a first sequence of the oligonucleotide probe; and
exposing the oligonucleotide probe to a second nucleic acid, different from the first nucleic acid and labeled with a second detection entity, such that at least a portion of the second nucleic acid hybridizes to a second sequence of the oligonucleotide probe.

2. The method of claim 1, wherein the act of exposing the oligonucleotide probe to the first nucleic acid and the act of exposing the oligonucleotide probe to the second nucleic acid occurs essentially simultaneously.

3. The method of claim 1, further comprising cleaving a precursor nucleic acid to produce the first nucleic acid and the second nucleic acid.

4. The method of claim 3, comprising:
separating the first nucleic acid and the second nucleic acid;
thereafter, labeling the first nucleic acid with the first detection entity and the second nucleic acid with the second detection entity.

5. The method of claim 3, further comprising identifying one or more restriction sites within the precursor nucleic acid by determining hybridization of the first nucleic acid with the oligonucleotide probe, and hybridization of the second nucleic acid with the oligonucleotide probe.

6. The method of claim 1, wherein the oligonucleotide probe is immobilized relative to a surface.

7. The method of claim 1, wherein the oligonucleotide probe has a length of at least 60 nucleotides.

8. The method of claim 1, wherein the first detection entity is fluorescent.

9. The method of claim 1, comprising providing a plurality of oligonucleotide probes, at least some of which are non-identical, and exposing the plurality of oligonucleotide probes to the first and second nucleic acids.

10. The method of claim 9, comprising providing at least 100 non-identical oligonucleotide probes.

11. The method of claim 9, wherein the plurality of oligonucleotide probes are each immobilized relative to a surface at a density of at least about 0.01 pmol/mm².

12. The method of claim 1, further comprising comparing hybridization of the first nucleic acid with the oligonucleotide probe, and hybridization of the second nucleic acid with the oligonucleotide probe.

13. The method of claim 12, further comprising determining a ratio of concentration of the first nucleic acid to the second nucleic acid.

14. An article, comprising:
a composition, constructed and arranged to be used in an assay of a sample comprising a first nucleic acid labeled with a first detection entity and a second nucleic acid, different from the first nucleic acid and labeled with a second detection entity, the composition comprising an oligonucleotide probe able to hybridize to at least portions of each of the first nucleic acid and the second acid such that each of the first detection entity and the second detection entity can be determined.

15. An article, comprising:
an array comprising a plurality of oligonucleotide probes, at least some of which each comprise a first nucleotide sequence able to hybridize to a first portion of a chromosome and a second nucleotide sequence able to hybridize to a second portion of the chromosome.

16. An article, comprising:
a composition comprising a first oligonucleotide probe comprising a first nucleotide sequence able to hybridize to a first portion of a first chromosome and a second nucleotide sequence able to hybridize to a second portion of the first chromosome, and a second oligonucleotide probe comprising a first nucleotide sequence able to hybridize to a first portion of a second chromosome and a second nucleotide sequence able to hybridize to a second portion of the second chromosome.

17. A method, comprising:
cleaving a nucleic acid into a plurality of nucleic acid fragments;
separating the nucleic acid fragments into at least a first sample and a second sample;
labeling at least some of the fragments of the first sample with a first detection entity and at least some of the fragments of the second sample with a second detection entity; and
exposing at least some of the nucleic acid fragments of the first and second samples to at least one compound probe.

18. A method, comprising:
cleaving a nucleic acid into a plurality of nucleic acid fragments, each having a first end and a second end;
labeling the first end of at least some of the nucleic acid fragments with a first detection label and the second end of at least some of the nucleic acid fragments with a second detection label; and
exposing at least some of the nucleic acid fragments to at least one compound probe.

19. A method, comprising:
labeling one or more portions of a first chromosome with a first detection entity and one or more portions of a second chromosome with a second detection entity;
fragmenting each of the first chromosome and the second chromosome to produce a plurality of chromosome fragments; and
exposing the chromosome fragments to one or more compound probes.

20. The method of claim 19, wherein the acts are performed in the order recited.

21. The method of claim 19, wherein at least some of the one or more compound probes comprises a first sequence able to hybridize the first chromosome but not the second chromosome, and a second sequence able to hybridize the second chromosome but not the first chromosome.

22. The method of claim 19, further comprising determining whether a compound probe of the one or more compound probes has hybridized to both a portion of a first chromosome and a portion of a second chromosome.

23. The method of claim 22, further comprising identifying a translocation between the first and second chromosomes based on hybridization of the compound probe.
